(19) United States
(12) Patent Application Publication
(10) Pub. No.: US 2026/0250346 A1
(43) Pub. Date: Aug. 27, 2026

(54) New hybrid protein CBD-HS-ES-GLP1, recombinant plasmid for its production, Escherichia coli producer strain and method OF PRODUCING GLP-1 polypeptide

(71) Applicants: SHEMYAKIN-OVCHINNIKOV INSTITUTE OF BIOORGANIC CHEMISTRY OF THE RUSSIAN ACADEMY OF SCIENCES, Russian Federation, Moscow (MX); AVVA PHARMACEUTICALS LTD, Limassol (CY)

(72) Inventors: Aleksey Alekseevich Zinchenko, Moscow (RU); Dmitriy Aleksandrovich MAKAROV, Moscow (RU); Igor Valentinovich MYAGKIKH, Moscow (RU); Daniil Mikhailovich PAVLENKO, Krasnogorsk (RU); Vasiliy Nikolaevich STEPANENKO, Moscow (RU)

(73) Assignees: AVVA PHARMACEUTICALS LTD, Limassol (CY); SHEMYAKIN-OVCHINNIKOV INSTITUTE OF BIOORGANIC CHEMISTRY OF THE RUSSIAN ACADEMY OF SCIENCES, Moscow (RU)

(21) Appl. No.: 18/992,560

(22) PCT Filed: Jul. 6, 2023
(86) PCT No.: PCT/RU2023/000203

§ 371 Date: Jan. 8, 2025

(30) Foreign Application Priority Data
Jul. 8, 2022 (RU) 202291910

(51) Int. Cl. C07K 14/605; C07K 19/00; C12N 15/70

(52) U.S. Cl. CPC C07K 14/605; C07K 19/00; C12N 15/70

(57) ABSTRACT

The invention relates to genetic and protein engineering and can be used in medicine and the pharmaceutical industry. Recombinant plasmid DNA pET23b-CBD-HS-ES-GLP1, pET-parB-CBD-HS-ES-GLP1 and pET23bKanR-CBD-HS-ES-GLP1 were constructed to provide the synthesis of a hybrid protein containing the GLP-1 polypeptide sequence in Escherichia coli cells. By transforming the E. coli BL21 (DE3) strain with plasmids pET23b-CBD-HS-ES-GLP1, pET-parB-CBD-HS-ES-GLP1 and pET23bKanR-CBD-HS-ES-GLP1, the CBD-HS-ES-GLP1 hybrid protein has been produced. A method of producing polypeptide GLP-1 has been developed, which involves preparing and cultivating a producer of a hybrid protein containing polypeptide GLP-1, followed by isolation and cleavage of the said hybrid protein. The invention enables the production of polypeptide GLP-1 with high yield and simplified technology.

### FIELD OF INVENTION

The present invention relates to the field of genetic and protein engineering and can be used in medicine and pharmaceutical industry. In particular, the present invention relates to a hybrid protein CBD-HS-ES-GLP1, intended for producing the GLP-1 polypeptide, recombinant plasmids pET23b-CBD-HS-ES-GLP1 for the expression of the specified hybrid protein, the strain-producer BL21 (DE3)/CBD-HS-ES-GLP1, producing the said hybrid protein, as well as a method for producing a GLP-1 polypeptide.

### PRIOR ART

The GLP-1 polypeptide, having the sequence of SEQ ID NO 1, is one of the incretins that lowers glucose levels by modulating insulin secretion by liver beta cells [Baggio LL, Drucker DJ (2007), Biology of incretins: GLP-1 and GIP. Gastroenterology 132:2131-2157]. GLP-1 and its analogs stimulate insulin secretion in patients with non-insulin-dependent diabetes. Moreover, GLP-1 and its analogs inhibit glucagon secretion, which leads to a significant decrease in blood glucose levels.

A known method of preparing a GLP-1 polypeptide using peptide synthesis [CA2778047, C07K14/605, publ. Jun. 15, 2000]. This method has all the disadvantages inherent in the chemical synthesis of polypeptides. Due to the low yield of the product, the production process requires a large amount of reagents, resulting in high costs and significant waste generation. The time-consuming nature of the method limits its effectiveness for large-scale production.

More promising is the preparation of the GLP-1 polypeptide using recombinant DNA technology. Thus, a method for producing glucagon-like polypeptides using a construct encoding a hybrid protein is known [U.S. Pat. No. 7,847,063, C12N15/8257, publ. Feb. 8, 2007]. The main disadvantage of this method is the use of cyanogen bromide, which makes it unsuitable for use as a pharmaceutical product due to the toxicity of cyanogen bromide and the difficulty of completely purifying the product from traces of this compound.

A known method for producing a GLP-1 polypeptide in the hybrid protein form that includes an affinity tag [U.S. Pat. No. 8,796,431, C12P21/06, publ. May 12, 2011]. Despite the simplicity of the purification procedure, achieved by using affinity chromatography, the final yield of the product is relatively low. In addition, acid hydrolysis is used in the hybrid protein cleavage stage leading to the excessive formation of by-products.

A known method for producing a GLP-1 polypeptide in a construct containing multiple copies of the corresponding gene [CA3057252, C07K14/605, publ. Sep. 27, 2018]. The disadvantage of this method is the use of two proteases at once at the step of hybrid protein cleavage, which increases the cost of the production process and complicates further purification. In addition, this method does not propose an approach to purification, which, given the features of the design used, will be complex and resource-intensive.

Closest to the described invention is a method of producing a GLP-1 polypeptide in a hybrid protein containing an intein sequence [WO2018/136572, C07K14/605, publ. Jul. 26, 2018]. This method uses a hybrid protein containing a chitin-binding domain, an intein, a spacer, a TEV proteinase recognition site, and a GLP-1 polypeptide. The main disadvantage of this method is the use of intein technology, which leads to a low yield of the final protein. This also uses the hybrid protein cleavage by TEV proteinase, leading to process complications and increasing the number of production stages. Another disadvantage is the lack of a complete purification scheme. The invention describes only the stage of purifying the hybrid protein from the producer cell proteins.

Thus, the technical problem solved by the present intention was to overcome the above disadvantages of the existing methods for producing a GLP-1 polypeptide, namely, to increase the efficiency and reduce the complexity and complexity of the method for producing a GLP-1 polypeptide, in particular, to increase the yield of the GLP-1 polypeptide, compared to methods known from the prior art. The technical result of the present invention is to achieve an increased yield of the GLP-1 polypeptide, which is at least 30% relative to the total cell protein, with a product purity of at least 95%. According to one particular embodiment of the invention, the product purity of 99% is achieved. According to one particular embodiment of the invention, the yield of GLP-1 polypeptide of 35% is achieved. According to another specific embodiment of the invention, the yield of the GLP-1 polypeptide of 40% is achieved.

This technical problem is solved by the present invention by providing a new hybrid protein CBD-HS-ES-GLP1, designed to prepare a GLP-1 polypeptide, a new recombinant plasmid pET23b-CBD-HS-ES-GLP1 for the expression of the specified hybrid protein, a new highly productive bacterial strain-a BL21 (DE3)/CBD-HS-ES-GLP1 producer that produces said hybrid protein; and a method for producing a GLP-1 polypeptide that makes it possible to prepare a GLP-1 polypeptide in high yield and high purity.

### SUMMARY OF INVENTION

The present invention relates to a hybrid protein CBD-HS-ES-GLP1 for producing a GLP-1 polypeptide having the amino acid sequence of SEQ ID NO 2 and containing the amino acid sequence X₁X₂YX₃, chitin-binding domain (CBD), enteropeptidase specific recognition site(ES), a hexahistidine domain (HS), a GLP-1 polypeptide having the amino acid sequence of SEQ ID NO 1, where X₁X₂YX₃ is an amino acid sequence in which X₁ is selected from the group consisting of A, R, N, D, C, Q, E, G, H, I, L, K, M, F, R, P, O, S, U, T, W, Y, and V, X₂ is selected from K or N, and X₃ missing or selected from H or Q.

In one embodiment of said hybrid protein, the amino acid sequence X₁X₂YX₃ is VKY.

In one embodiment of said hybrid protein, the amino acid sequence X₁X₂YX₃ is VNY.

The present invention also relates to recombinant plasmid DNA pET23b-CBD-HS-ES-GLP1 for expression of said CBD-HS-ES-GLP1 hybrid protein, consisting of the following key genetic elements:
T7 promoter and lacO operator;
a synthetic nucleotide sequence shown in SEQ ID NO 5 encoding a hybrid protein CBD-HS-ES-GLP1 according to the present invention having the amino acid sequence of SEQ ID NO 2;
antibiotic resistance gene for selection of recombinant cells;
origin of DNA replication of bacteriophage f1 (f1 ori).

In one embodiment, said recombinant plasmid DNA pET23b-CBD-HS-ES-GLP1 is 3953 bp long, contains an antibiotic resistance gene an ampicillin resistance gene (AmpR), and a bacterial ampicillin resistance gene promoter (AmpR promoter), and has an arrangement of elements as shown in FIG. 1.

In another embodiment, said pET23b-CBD-HS-ES-GLP1 recombinant plasmid DNA is 3820 bp long, contains the antibiotic resistance gene kanamycin (KanR) as an antibiotic resistance gene, and has an arrangement as shown in FIG. 2. Per the present invention, the plasmid DNA in this embodiment is also known as pET23bKanR-CBD-HS-ES-GLP1.

In another embodiment, said recombinant plasmid DNA pET23b-CBD-HS-ES-GLP1 is recombinant plasmid DNA pET-parB-CBD-HS-ES-GLP1, has a length of 4391 bp, contains a hok/sok module that stabilizes plasmids [T, Gerdes K. Mechanism of post-segregational killing by the hok/sok system of plasmid R1. Sok antisense RNA regulates hok gene expression indirectly through the overlapping mok gene. J Mol Biol. 1992 Jan. 5; 223 (1): 41-54. doi: 10.1016/0022-2836 (92) 90714-u. PMID: 1370544] and has an arrangement of elements as shown in FIG. 3.

The present invention also relates to Escherichia coli strain BL21 (DE3)/CBD-HS-ES-GLP1 producing hybrid protein CBD-HS-ES-GLP1 (SEQ ID NO 2) and containing said recombinant DNA plasmid pET23b-CBD-HS-ES-GLP1.

In another embodiment, the specified strain of Escherichia coli BL21 (DE3)/CBD-HS-ES-GLP1 contains a recombinant DNA plasmid pET23b-CBD-HS-ES-GLP1, which has a length of 3953 bp, contains the ampicillin resistance gene (AmpR) as an antibiotic resistance gene and the bacterial promoter of the ampicillin resistance gene (AmpR promoter); FIG. 1 shows the location of its genetic elements.

In one embodiment, the specified strain of Escherichia coli BL21 (DE3)/CBD-HS-ES-GLP1 contains a recombinant DNA plasmid pET23b-CBD-HS-ES-GLP1, which has a length of 3820 bp, contains the kanamycin resistance gene (KanR) as an antibiotic resistance gene; FIG. 2 shows the location of its genetic elements. According to the present invention, the Escherichia coli strain according to this embodiment is also referred to as BL21 (DE3)/KanR/CBD-HS-ES-GLP1.

In another embodiment, said recombinant plasmid DNA pET-parB-CBD-HS-ES-GLP1 has a length of 4391 bp, contains a hok/sok module that stabilizes plasmids [T, Gerdes K. Mechanism of post-segregational killing by the hok/sok system of plasmid R1. Sok antisense RNA regulates hok gene expression indirectly through the overlapping mok gene. J Mol Biol. 1992 Jan. 5; 223 (1): 41-54. doi: 10.1016/0022-2836 (92) 90714-u. PMID: 1370544] and has an arrangement of elements as shown in FIG. 3. According to the present invention, the Escherichia coli strain according to this embodiment is also referred to as BL21 (DE3)/parB/CBD-HS-ES-GLP1. According to the present invention, Escherichia coli strain BL21 (DE3)/CBD-HS-ES-GLP1 is obtained by transforming cells of Escherichia coli strain BL21 (DE3) with a recombinant plasmid, according to the present invention.

The present invention also relates to a method for producing a GLP-1 polypeptide having the amino acid sequence of SEQ ID NO 1, comprising the stages:
a) cultivating the Escherichia coli BL21 (DE3)/CBD-HS-ES-GLP1 producer strain according to the present invention to prepare cell biomass of the producer strain for the production of CBD-HS-ES-GLP1 hybrid protein, according to the present invention;
b) isolating the hybrid protein CBD-HS-ES-GLP1 according to the present invention from the cell biomass of the producer strain obtained at stage (a);
c) enzymatic cleavage of the CBD-HS-ES-GLP1 hybrid protein obtained at stage (b) to form a GLP-1 polypeptide with the amino acid sequence of SEQ ID NO 1;
d) purification of the GLP-1 polypeptide obtained at stage (c).

In one embodiment of the present invention, at stage (b), the cells are separated from the culture fluid, the cells are disintegrated, the inclusion bodies are isolated from the resulting disintegrate, and the inclusion bodies are solubilized.

In another embodiment, at stage (b), when separating the inclusion bodies from the resulting disintegrate, ammonium sulphate is added.

In another embodiment of the present invention, at stage (a), the cultivation of Escherichia coli BL21 (DE3)/CBD-HS-ES-GLP1 cells is carried out in a growth medium for at least 7 hours.

In another embodiment, at stage (a), the induction of recombinant protein biosynthesis by Escherichia coli BL21 (DE3)/CBD-HS-ES-GLP1 cells is carried out 3 hours after the start of cultivation with isopropyl-β-D-1-thiogalactopyranoside.

In another embodiment of the present invention, at stage (c), the hybrid protein cleavage is carried out with an enteropeptidase.

In another embodiment of the present invention, at stage (d), the purification of the GLP-1 polypeptide is carried out using a single chromatography stage.

In another embodiment of the present invention, at stage (d), the purification of the GLP-1 polypeptide is carried out using several chromatography stages.

In another embodiment, at stage (d), the purification of the CBD-HS-ES-GLP1 hybrid protein is carried out chromatographically on a metal chelate sorbent.

In another embodiment, at stage (b), the purification of the CBD-HS-ES-GLP1 hybrid protein is carried out chromatographically on a cation exchange sorbent.

In another embodiment, at stage (b), the purification of the CBD-HS-ES-GLP1 hybrid protein is carried out chromatographically on a chitin sorbent.

In another embodiment, at stage (d), the purification of the GLP-1 polypeptide is carried out using reverse-phase chromatography.

In another embodiment, at stage (d), the purification of the GLP-1 polypeptide is carried out using size exclusion chromatography.

In another embodiment, at stage (d), the purification of the GLP-1 polypeptide is carried out by precipitation with ammonium sulphate and extraction of impurities with a buffer solution.

In another embodiment, at stage (d), the purification of the CBD-HS-ES-GLP1 hybrid protein is carried out chromatographically on a chitin sorbent.

The present inventors have surprisingly found that their uniquely structured CBD-HS-ES-GLP1 hybrid protein and pET23b-CBD-HS-ES-GLP1 plasmid DNA for expression of said hybrid protein achieve an increased yield of the GLP-1 polypeptide of at least 30% relative to the total cell protein with a preparation purity of at least 95% in the course of the new method of producing the GLP-1 polypeptide developed by the authors of the present invention. The specified technical result is fully confirmed by the Examples given in this document below.

### BRIEF DESCRIPTION OF THE DRAWINGS

FIG. 1. Plasmid map; legends:
fl ori—origin of DNA replication of bacteriophage f1,
AmpR—ampicillin resistance gene (β-lactamase gene),
ori—origin of replication colE1,
T7 promoter—promoter of bacteriophage T7,
CBD-HS-ES-GLP1—CBD-HS-ES-GLP1 hybrid protein gene, including GLP-1 polypeptide, hexahistidine and chitin-binding sequences,
T7 terminator—terminator of bacteriophage T7.

FIG. 2. Plasmid map; legends:
fl ori—origin of DNA replication of bacteriophage f1,
KanR—kanamycin resistance gene (aminoglycoside phosphotransferase gene),
ori—origin of replication colE1,
T7 promoter—promoter of bacteriophage T7,
CBD-HS-ES-GLP1—CBD-HS-ES-GLP1 hybrid protein gene, including GLP-1 polypeptide, hexahistidine and chitin-binding sequences,
T7 terminator—terminator of bacteriophage T7.

FIG. 3. Plasmid map; legends:
f1 ori—origin of DNA replication of bacteriophage f1,
KanR—kanamycin resistance gene (aminoglycoside phosphotransferase gene),
ori—origin of replication colE1,
T7 promoter—promoter of bacteriophage T7,
parB—hok/sok locus
CBD-HS-ES-GLP1—CBD-HS-ES-GLP1 hybrid protein gene, including GLP-1 poly-peptide, hexahistidine and chitin-binding sequences,
T7 terminator—terminator of bacteriophage T7.

FIG. 4. Electrophoretic analysis of the total cell lysate.
1—at the moment of induction
2—2 hours after induction
3—4 hours after induction
4—5 hours after induction
5—molecular weight standards, Pierce Unstained Protein MW Marker, Thermo, cat #26610

FIG. 5. Electrophoretic analysis of the hybrid protein CBD-HS-ES-GLP1.
1—molecular weight standards, Pierce Unstained Protein MW Marker, Thermo, cat #26610
2 to 3-hybrid protein CBD-HS-ES-GLP1 before incubation with enteropeptidase
4 to 5—the reaction mixture containing the GLP-1 polypeptide obtained after incubation with enteropeptidase

FIG. 6. Chromatogram obtained during the chromato-mass-spectrometric analysis of the GLP-1 polypeptide.

FIG. 7. The mass spectrum obtained during gas chromatography-mass spectrometric analysis of the GLP-1 polypeptide.

FIG. 8. Electrophoretic analysis of the total cell lysate.
1—culture before induction
2—at the moment of induction
3—4 hours after induction
4—molecular weight standards, Pierce Unstained Protein MW Marker, Thermo, cat #26610

FIG. 9. Electrophoretic analysis of the total cell lysate, where the given designations have the following meanings: 1—molecular weight standards, Pierce Unstained Protein MW Marker, Thermo, cat #26610, 2—1 hour after induction, 3—2 hours after induction, 4—4 hours after induction.

### DETAILED DESCRIPTION OF INVENTION

The present invention relates to a hybrid protein CBD-HS-ES-GLP1 to prepare a GLP-1 polypeptide having the amino acid sequence of SEQ ID NO 2, containing the amino acid sequence X₁X₂YX₃, chitin-binding domain (CBD), enteropeptidase specific recognition site(ES), a hexahistidine domain (HS), a GLP-1 polypeptide having the amino acid sequence of SEQ ID NO 1, where X₁X₂YX₃ is an amino acid sequence in which X₁ is selected from the group consisting of A, R, N, D, C, Q, E, G, H, I, L, K, M, F, R, P, O, S, U, T, W, Y, and V, X₂ is selected from K or N, and X₃ missing or selected from H or Q.

The present invention also relates to recombinant pET23b-CBD-HS-ES-GLP1 plasmid DNA for expression of said CBD-HS-ES-GLP1 hybrid protein, consisting of the following key genetic elements:
T7 promoter and lacO operator;
a synthetic nucleotide sequence shown in SEQ ID NO 5 encoding a hybrid protein according to the present invention having the amino acid sequence of SEQ ID NO 2;
antibiotic resistance gene for selection of recombinant cells;
origin of DNA replication of bacteriophage f1 (f1 ori).

In one embodiment, said recombinant plasmid DNA pET23b-CBD-HS-ES-GLP1 is 3953 bp long, contains as antibiotic resistance gene the antibiotic resistance gene ampicillin (AmpR) and the bacterial promoter of the ampicillin resistance gene (AmpR promoter); FIG. 1 shows the location of its genetic elements.

In another embodiment, said recombinant plasmid DNA pET23b-CBD-HS-ES-GLP1 is 3820 bp long and contains the antibiotic resistance gene kanamycin (KanR) as an antibiotic resistance gene; FIG. 2 shows the location of its genetic elements. Per the present invention, the plasmid DNA in this embodiment is also known as pET23bKanR-CBD-HS-ES-GLP1.

In another embodiment, said recombinant plasmid DNA pET-parB-CBD-HS-ES-GLP1 is 4391 bp long, contains a plasmid stabilizing hok/sok module, and has an arrangement of elements as shown in FIG. 3. According to the present invention, the plasmid DNA according to this embodiment is also referred to as pET-parB-CBD-HS-ES-GLP1.

The present invention also relates to Escherichia coli strain BL21 (DE3)/CBD-HS-ES-GLP1 producing hybrid protein CBD-HS-ES-GLP1 (SEQ ID NO 2) containing said recombinant DNA plasmid pET23b-CBD-HS-ES-GLP1.

In one embodiment, the specified strain of Escherichia coli BL21 (DE3)/CBD-HS-ES-GLP1 contains a recombinant DNA plasmid pET23b-CBD-HS-ES-GLP1, which has a length of 3953 bp and contains as a resistance gene to the antibiotic resistance gene ampicillin (AmpR) and the bacterial promoter of the ampicillin resistance gene (AmpR promoter); FIG. 1 shows the location of its genetic elements.

In one embodiment, the specified strain of Escherichia coli BL21 (DE3)/CBD-HS-ES-GLP1 contains a recombinant DNA plasmid pET23b-CBD-HS-ES-GLP1, which has a length of 3820 bp, contains the kanamycin resistance gene (KanR) as an antibiotic resistance gene;

FIG. 2 shows the location of its genetic elements. According to the present invention, the Escherichia coli strain according to this embodiment is also referred to as BL21 (DE3)/KanR/CBD-HS-ES-GLP1.

In one embodiment, said strain of Escherichia coli BL 21 (DE3)/CBD-HS-ES-GLP1 contains a recombinant DNA plasmid pET-parB-CBD-HS-ES-GLP1, which has a length of 4391 bp, contains the module hok/sok stabilizing plasmids, and the location of its genetic elements is shown in FIG. 3. According to the present invention, the Escherichia coli strain according to this embodiment is also referred to as BL21 (DE3)/parB/CBD-HS-ES-GLP1.

According to the present invention, Escherichia coli strain BL21 (DE3)/CBD-HS-ES-GLP1 is obtained by transforming cells of Escherichia coli strain BL21 (DE3) with a recombinant plasmid, according to the present invention.

Another object of the present invention is a method of producing a GLP-1 polypeptide (SEQ ID NO 1), which includes the stages:
a) cultivating the Escherichia coli BL21 (DE3)/CBD-HS-ES-GLP1 producer strain according to the present invention to prepare cell biomass of the producer strain producing the CBD-HS-ES-GLP1 hybrid protein, according to the present invention;
b) isolating the hybrid protein CBD-HS-ES-GLP1 according to the present invention from the cell biomass of the producer strain obtained at stage (a);
c) enzymatic cleavage of the CBD-HS-ES-GLP1 hybrid protein obtained at stage (b) to form a GLP-1 polypeptide with the amino acid sequence of SEQ ID NO 1;
d) purification of the GLP-1 polypeptide obtained at stage (c).

At stage b) the cells are separated from the culture fluid, the cells are disintegrated, the inclusion bodies are isolated from the obtained disintegrate, and the inclusion bodies are solubilized.

Cultivation of Escherichia coli BL21 (DE3)/CBD-HS-ES-GLP1 cells in a growth medium is carried out for at least 6 hours.

Induction of recombinant protein biosynthesis by Escherichia coli BL21 (DE3)/CBD-HS-ES-GLP1 cells is carried out 2 to 5 hours after the start of cultivation using isopropyl-β-D-1-thiogalactopyranoside.

Separation of the culture fluid from the cells is carried out by centrifugation.

Cell disintegration is carried out using an ultrasonic disintegrator.

The isolation of inclusion bodies from the disintegrate is carried out by centrifugation.

Isolating the hybrid protein CBD-HS-ES-GLP1 from the resulting solubilized inclusion bodies is carried out chromatographically on a metal-chelate, chitin, or ion-exchange sorbent.

### Definitions and Terms

Various terms relating to the subject matter of the present invention are used above, as well as in the description and claims. Unless otherwise specified, all technical and scientific terms used in this application have the same meaning as understood by those skilled in the art. References to techniques used in the description of this invention refer to well-known methods, including modifications of these methods and replacing them with equivalent methods known to those skilled in the art.

In the description of the present invention, the terms “comprises” and “comprising” are interpreted to mean “includes, among other things”. These terms are not intended to be construed as “consisting only of”.

As used herein, “GLP-1 polypeptide” refers to a peptide with the following amino acid sequence of SEQ ID NO 1.

The term “enteropeptidase” in this document means a proteolytic enzyme with the EC code 3.4.21.9, as well as all its fragments and analogs that have specific proteolytic activity against segments of amino acid sequences, including-Asp-Asp-Asp-Asp-Lys-(SEQ ID NO 6) and -Asp-Asp-Asp-Asp-Arg-(SEQ ID NO 4).

### Embodiments of the Present Invention

The following will be given embodiments of the invention in several examples. Each example is provided by an explanation of the invention and should not be considered a limitation thereof. Those skilled in the art will appreciate that various modifications and changes may be made to the invention without departing from the scope or spirit thereof. For example, features shown or described as one embodiment may be used in another embodiment to prepare yet another embodiment.

Thus, modifications and changes falling within the scope of the appended paragraphs and their equivalents are intended to be covered by the present invention. When providing ranges for parameters, it is assumed that the range also includes each of the endpoints of that range. It should be understood that this implementation describes only exemplary embodiments and is not intended to limit the broader aspects of the invention. These broader aspects are implemented through exemplary circuits, structures, techniques, and procedures.

### Creation of Expression Plasmid

The object of this invention is solved by constructing the expression plasmid pET23b-CBD-HS-ES-GLP1 with a length of 3953 bp, which ensures the expression of the hybrid protein CBD-HS-ES-GLP1 in Escherichia coli cells transformed with this plasmid.

This plasmid, referred to as pET23b-CBD-HS-ES-GLP1, consists of the following key genetic elements arranged according to FIG. 1:
ampicillin resistance gene (AmpR) and bacterial promoter of ampicillin resistance gene (AmpR promoter);
origin of DNA replication of bacteriophage f1 (f1 ori);
T7 promoter and lacO operator;
a synthetic nucleotide sequence shown in SEQ ID NO 5 encoding a hybrid protein CBD-HS-ES-GLP1 (SEQ ID NO 2) containing a GLP-1 polypeptide (SEQ ID NO 1), an enterokinase recognition site of SEQ ID NO 4, a leader polypeptide (SEQ ID NO 3), which has an HS hexahistidine site and a CBD chitin-binding site.

In the above plasmid, the structure of which is disclosed in FIG. 1 of the present description, the AmpR gene is intended for selecting stable Escherichia coli cells. The bacterial promoter of the ampicillin resistance gene (AmpR promoter) is intended for its expression.

The origin of replication of bacteriophage fl (Analysis of Genes and Genomes, John Wiley & Sons, 2004, S. 140) is widely used to create expression vectors.

The translation product of the synthetic sequence SEQ ID NO 5 is a polypeptide of the sequence SEQ ID NO 2, including the leader polypeptide SEQ ID NO 3, having an HS hexahistidine site and a CBD chitin-binding site, a human enteropeptidase recognition site ES (SEQ ID NO 4), a GLP-1 polypeptide.

The structure of said plasmid DNA (plasmid) consisting of the specified key genetic elements is shown in FIG. 1.

According to the invention, the plasmid is obtained from the plasmid vector pET-23b described in the prior art (https://www.merckmillipore.com/RU/ru/product/pET-23b+-DNA-Novagen,EMD_BIO-69746). To prepare a plasmid according to the invention, the sequence of SEQ ID NO 5, obtained by total nucleotide synthesis, is inserted into the plasmid vector pET-23b at the XhoI and NdeI restriction sites.

Moreover, for the present invention, to create the plasmid shown in FIG. 1, other genetic engineering approaches, currently known in the art or which will be created later, not explicitly mentioned in this specification, can be used. In implementing specific embodiments of this invention, a specialist can choose the most optimal method for creating a plasmid based on their existing knowledge.

Creation of Expression Plasmid with a Hok/Sok Stabilizing Module

The stated technical problem of the present invention can also be solved by constructing the expression plasmid pET-parB-CBD-HS-ES-GLP1 with a length of 4391 bp, which ensures the expression of the hybrid protein CBD-HS-ES-GLP1 in Escherichia Coli cells, transformed with the said plasmid.

The said plasmid, hereinafter referred to as pET-parB-CBD-HS-ES-GLP1, consists of the following key genetic elements arranged according to FIG. 3:
kanamycin resistance gene (KanR) and bacterial kanamycin resistance gene promoter;
origin of bacteriophage replication f1 (f1 ori);
T7 promoter and lacO operator;
parB-hok/sok locus;
synthetic nucleotide sequence shown in SEQ ID NO 5 encoding a CBD-HS-ES-GLP1 hybrid protein (SEQ ID NO 2) containing polypeptide GLP-1 (SEQ ID NO 1), enterokinase recognition site of SEQ ID NO 4, leader polypeptide (SEQ ID NO 3) containing a HS hexahistidine site and a CBD chitin binding site.

In the said plasmid, the structure of which is disclosed in FIG. 3 of the present description, the KanR gene is intended for selection of stable Escherichia Coli cells. The bacterial promoter of the kanamycin resistance gene is intended for its expression. The hok/sok locus is designed to increase the stability of the plasmid, including the cultivation of producer cells in a large volume without the use of antibiotics.

The translation product of the synthetic sequence SEQ ID NO 5 is polypeptide of the sequence SEQ ID NO 2, including the leader polypeptide SEQ ID NO 3, having HS hexahistidine site and CBD chitin binding site, human enteropeptidase recognition site ES (SEQ ID NO 4), polypeptide GLP-1.

The structure of said plasmid DNA (plasmid) consisting of said key genetic elements is shown in FIG. 3.

The plasmid according to the invention is obtained from the pET plasmid vector. In this vector, the kanamycin antibiotic resistance gene known in the art is inserted preferably, but not limited, at the PciI and PsiI restriction sites to obtain the pET/KanR plasmid. The sequence of SEQ ID NO 7 containing the prior art hok/sok locus is preferably, but not limited to, inserted into the pET/KanR plasmid vector at the PciI and XmaI restriction sites to obtain the pET/parB plasmid. To obtain a plasmid according to the invention, the sequence of SEQ ID NO 5 obtained by total nucleotide synthesis is inserted into the pET/parB plasmid vector at the XhoI and NdeI restriction sites.

Also for the purposes of the present invention, to create the plasmid shown in FIG. 3, other genetic engineering techniques not explicitly mentioned in this specification that are currently known in the art or will be created later may be used. In the implementation of specific embodiments of the present invention, the specialist, based on the existing level of knowledge, can choose the most optimal method of creating a plasmid.

Creation of an Expression Plasmid with a Kanamycin Resistance Gene

The stated technical problem of the present invention can also be solved by constructing the expression plasmid pET23bKanR-CBD-HS-ES-GLP1 with a length of 3820 bp, which ensures the expression of the hybrid protein CBD-HS-ES-GLP1 in Escherichia coli cells transformed with this plasmid.

This plasmid, hereinafter referred to as pET23bKanR-CBD-HS-ES-GLP1, consists of the following key genetic elements arranged according to FIG. 2:
kanamycin resistance gene (KanR) and bacterial kanamycin resistance gene promoter;
origin of DNA replication of bacteriophage f1 (f1 ori);
T7 promoter and lacO operator;
a synthetic nucleotide sequence shown in SEQ ID NO 5 encoding a hybrid protein CBD-HS-ES-GLP1 (SEQ ID NO 2) containing a GLP-1 polypeptide (SEQ ID NO 1), an enterokinase recognition site of SEQ ID NO 4, a leader polypeptide (SEQ ID NO 3), which has an HS hexahistidine site and a CBD chitin-binding site.

In the above plasmid, the structure of which is disclosed in FIG. 2 of the present description, the KanR gene is for selecting stable Escherichia coli cells. The bacterial promoter of the kanamycin resistance gene is intended for its expression.

The translation product of the synthetic sequence SEQ ID NO 5 is a polypeptide of the sequence SEQ ID NO 2, including the leader polypeptide SEQ ID NO 3, having an HS hexahistidine site and a CBD chitin-binding site, a human enteropeptidase recognition site ES (SEQ ID NO 4), a GLP-1 polypeptide.

The structure of the specified plasmid DNA (plasmid) consisting of the said key genetic elements is shown in FIG. 2.

The plasmid according to the invention is obtained from the plasmid vector pET-23b. In this vector, the kanamycin antibiotic resistance gene known from the prior art (https://plasmid.med.harvard.edu/PLASMID/GetVectorDetail.do?vectorid=319 #: ~: text=Name % 3A-,pET28a,pET28% 2C % 20pET %2D28a) is inserted preferably, but without limitation, at the restriction sites PciI and PsiI to prepare plasmid pET23b/KanR. According to the invention, to prepare a plasmid, the sequence of SEQ ID NO 5, obtained by total nucleotide synthesis, is inserted into the plasmid vector pET-23b/KanR at the XhoI and NdeI restriction sites.

Moreover, for the present invention, to create the plasmid shown in FIG. 2, other genetic engineering techniques not explicitly mentioned herein, which are currently known in the art or will be developed later, may be used. In implementing specific embodiments of this invention, a specialist can choose the most optimal method for creating a plasmid based on their existing knowledge.

Preparation of the Producer Strain E. coli BL21 (DE3)/CBD-HS-ES-GLP1

The BL21 (DE3)/CBD-HS-ES-GLP1 strain is obtained by transforming Escherichia coli BL21 (DE3) cells with the 3953 bp pET23b-CBD-HS-ES-GLP1 expression plasmid encoding the hybrid protein SEQ ID NO 2, consisting of the following key genetic elements:
ampicillin resistance gene (AmpR) and bacterial promoter of ampicillin resistance gene (AmpR promoter);
origin of bacteriophage fl replication;
T7 promoter and lacO operator;
a synthetic nucleotide sequence shown in SEQ ID NO 5 encoding a hybrid protein CBD-HS-ES-GLP1 (SEQ ID NO 2) containing a GLP-1 polypeptide (SEQ ID NO 1), an enterokinase recognition site of SEQ ID NO 4, a leader polypeptide (SEQ ID NO 3), which has an HS hexahistidine site and a CBD chitin-binding site.

According to the invention, the starting material for creating a producer strain is a strain of E. coli BL21 (DE3) known from the prior art (Haeyoung Jeong, Valérie Barbe, Choong Hoon Lee, David Vallenet, Dong Su Yu, Sang-Haeng Choi, Arnaud Couloux, Seung-Won Lee, Sung Ho Yoon, Laurence Cattolico, Cheol-Goo Hur, Hong-Seog Park, Béatrice Segurens, Sun Chang Kim, Tae Kwang Oh, Richard E. Lenski, F. William Studier, Patrick Daegelen, Jihyun F. Kim, Genome Sequences of Escherichia coli B strains REL606 and BL21 (DE3), Journal of Molecular Biology, Volume 394, Issue 4, 2009, 644-652). Expression plasmid with a length of 3953 bp, consisting of key genetic elements (a) to (e), located relative to each other as shown in FIG. 1, transform cells of the strain E. coli BL21 (DE3).

Preferably (without limitation), to introduce the specified plasmid into the cells of the strain E. coli BL21 (DE3), use the method of electroporation known from the prior art (Tamara Kleber-Janke, Wolf-Meinhard Becker, Use of Modified BL21 (DE3) Escherichia coli Cells for High-Level Expression of Recombinant Peanut Allergens Affected by Poor Codon Usage, Protein Expression, and Purification, Volume 19, Issue 3, 2000, 419-424) and incorporated herein by reference. The skilled specialist will appreciate that other embodiments of the present invention may use other transformation methods known in the art to introduce the plasmid into E. coli BL21 (DE3) cells, e. g., polyethylene glycol method and calcium chloride method. Also for the present invention, for introduction into E. coli BL21 (DE3) cells, the plasmid shown in FIG. 1, and other transformation methods not explicitly mentioned in the present description, which are currently known in the prior art or will be created subsequently, can be used. In implementing specific embodiments of this invention, a specialist can choose the most optimal method for cell transformation based on their existing knowledge.

Transformed cells are plated on Petri dishes with agar medium supplemented with the selection agent ampicillin to a final ampicillin concentration of 50 μg/mL of a cell. The pET23b-CBD-HS-ES-GLP1 plasmid DNA was isolated from the ampicillin-resistant clones and analyzed by sequencing.

The proposed strain-producer Escherichia coli BL21 (DE3)/CBD-HS-ES-GLP1 is characterized by the following features:

Morphological features: rod-shaped cells, gram-negative, non-spore-bearing.

Cultural features: cells grow well on simple nutrient media; when growing on agar medium “LB” (per 1 1-10 g peptone, 5 g yeast extract, 10 g NaCl, 20 g agar)—colonies are round, smooth, cloudy, shiny, gray, and the edges are even.

Physical and biological signs: cells grow at 4° C. to 40° C. with an optimal pH value of 6.8 to 7.5. As a source of nitrogen, both mineral salts in the ammonium form and organic compounds, including peptone, tryptone, yeast extract, amino acids, etc., are used. Amino acids, glycerol, and carbohydrates are used as a carbon source.

Antibiotic resistance: cells show resistance to penicillin antibiotics (up to 500 μg/mL).

Preparation of the Producer Strain E. coli BL21 (DE3)/KanR/CBD-HS-ES-GLP1

The BL21 (DE3)/KanR/CBD-HS-ES-GLP1 strain is obtained by transforming Escherichia coli BL21 (DE3) cells with the 3820 bp pET23bKanR-CBD-HS-ES-GLP1 expression plasmid encoding the hybrid protein SEQ ID NO 2, consisting of the following key genetic elements:
kanamycin resistance gene (KanR) and bacterial kanamycin resistance gene promoter;
origin of bacteriophage fl replication;
T7 promoter and lacO operator;
a synthetic nucleotide sequence shown in SEQ ID NO 5 encoding a hybrid protein (SEQ ID NO 2) containing a GLP-1 polypeptide (SEQ ID NO 1), an enterokinase recognition site of SEQ ID NO 4, a leader polypeptide (SEQ ID NO 3), which has an HS hexahistidine site and a CBD chitin-binding site.

According to the invention, the starting material for creating a producer strain is a strain of E. coli BL21 (DE3) known from the prior art. Expression plasmid length 3820 bp, consisting of key genetic elements (a) to (e), located relative to each other as shown in FIG. 2, transform cells of the E. coli BL21 (DE3) strain.

Preferably (without limitation), for the introduction of the specified plasmid in the cells of the strain E. coli BL21 (DE3) use the method of electroporation, known from the prior art and included in the present description by reference. The skilled specialist will appreciate that other embodiments of the present invention may use other transformation methods known in the art to introduce the plasmid into E. coli BL21 (DE3) cells, e. g., polyethylene glycol method and calcium chloride method. Moreover, for the present invention, for introduction into E. coli BL21 (DE3) cells, the plasmid shown in FIG. 2, and other transformation methods not explicitly mentioned in the present description, which are currently known in the prior art or will be created subsequently, can be used. In implementing specific embodiments of this invention, a specialist can choose the most optimal method for cell transformation based on their existing knowledge.

Transformed cells are plated on Petri dishes with agar medium supplemented with the selection agent ampicillin to a final ampicillin concentration of 50 μg/mL of a cell. The pET23bKanR-CBD-HS-ES-GLP1 plasmid DNA was isolated from the ampicillin-resistant clones and analyzed by sequencing.

The proposed strain-producer Escherichia coli BL21 (DE3)/KanR/CBD-HS-ES-GLP1 is characterized by the following features:

Morphological features: rod-shaped cells, gram-negative, non-spore-bearing.

Cultural characteristics: cells grow well on simple nutrient media. When growing on agar medium “LB” (per 1 1-10 g peptone, 5 g yeast extract, 10 g NaCl, 20 g agar)—the colonies are round, smooth, cloudy, shiny, and gray, and the edges are even.

Physical and biological signs: cells grow at 4° C. to 40° C. with an optimal pH value of 6.8 to 7.5. As a source of nitrogen, both mineral salts in the ammonium form and organic compounds, including peptone, tryptone, yeast extract, amino acids, etc., are used. Amino acids, glycerol, and carbohydrates are used as a carbon source.

Antibiotic resistance: cells show resistance to kanamycin (up to 500 μg/mL).

### Preparation, Isolation, and Purification of the Hybrid Protein CBD-HS-ES-GLP1

The method for producing the CBD-HS-ES-GLP1 hybrid protein includes cultivating cells of the Escherichia coli BL21 (DE3)/CBD-HS-ES-GLP1 producer strain obtained by transforming Escherichia coli BL21 (DE3) cells disclosed in FIG. 1 with a 3953 bp plasmid consisting of the indicated key genetic elements, on a growth medium to prepare a culture fluid, separation of cell biomass from the culture fluid, cell disintegration, isolation of inclusion bodies from the resulting disintegrate, solubilization of inclusion bodies and isolation of the hybrid protein CBD-HS-ES-GLP1 from the resulting solubilized inclusion bodies.

Another method for producing the CBD-HS-ES-GLP1 hybrid protein involves culturing cells of the Escherichia coli BL21 (DE3)/KanR/CBD-HS-ES-GLP1 producer strain obtained by transforming Escherichia coli BL21 (DE3) cells disclosed in FIG. 2 with a plasmid 3820 bp long, consisting of the indicated key genetic elements, on a growth medium to prepare a culture fluid, separation of cell biomass from the culture fluid, cell disintegration, isolation of inclusion bodies from the obtained disintegrate, solubilization of inclusion bodies and isolation of the hybrid protein CBD-HS-ES-GLP1 from the resulting solubilized inclusion bodies.

In a preferred embodiment, but without limitation, Escherichia coli BL21 (DE3)/CBD-HS-ES-GLP1 cells are cultured in a growth medium for at least 7 hours.

In a preferred embodiment, but without limitation, the induction of recombinant protein biosynthesis by Escherichia coli BL21 (DE3)/CBD-HS-ES-GLP1 cells is carried out at 3 hours of culture with isopropyl-β-D-1-thiogalactopyranoside.

In a preferred embodiment, but without limitation, the separation of the culture fluid from the cells is carried out by centrifugation.

In a preferred embodiment, but without limitation, cell disintegration is carried out using an ultrasonic disintegrator.

In a preferred embodiment, but without limitation, the separation of inclusion bodies from the disintegrate is carried out by centrifugation.

In a preferred embodiment, but also without limitation, the isolation of the CBD-HS-ES-GLP1 hybrid protein from the obtained solubilized inclusion bodies is carried out chromatographically. In more preferred embodiments, but not limited to, the isolation of the CBD-HS-ES-GLP1 hybrid protein from the solubilized inclusion bodies is carried out on a metal chelate chromatographic sorbent.

Cleavage of the hybrid protein CBD-HS-ES-GLP1 in a preferred embodiment, but also without limitation, is carried out using enteropeptidase, based on the ratio of 1 enzyme unit per 1 mg of hybrid protein.

Finally, in a preferred embodiment, but also without limitation, further purification of the GLP-1 polypeptide is carried out using chromatographic methods.

The implementation of the claimed invention is illustrated by the following examples, which do not limit the scope of the present invention.

### Examples

### Example 1 Construction of the Expression Plasmid

Chemical synthesis of oligonucleotides is performed by the solid-phase phosphoramidite method on an ASM-102U DNA synthesizer (BIOSSET, Novosibirsk) with the growth of the oligonucleotide chain in the direction from the 3′-end to the 5′-end using protected phosphoramidites—5′-dimethoxytrityl-N-acyl-2′-deoxynucleoside-3′-O-(β-cyanoethyl-diisopropylamino)-phosphites activated with tetrazole. The synthesis is carried out on a scale of 0.5 to 0.7 μmol, using porous glass as a carrier (pore size of 500 Å), to which the first nucleoside unit is attached via a 3′-succinate bond (load 20 to 30 μmol/g). A synthetic run of the standard phosphoramidite method is used.

To prepare the DNA vector, plasmids pET23b (450 μg, 150 μmol) are treated in 6 mL of buffer with 20 mM Tris-acetate, 10 mM magnesium acetate, 50 mM potassium acetate, 100 μg/mL BSA restrictase XhoI (1000 active units), and then, in 6 mL of buffer 100 mM NaCl, 50 mM Tris-HCl, 10 mM MgCl₂, 100 μg/mL BSA with NdeI restrictase (1000 active units) for 1 hour at 37° C. The vector fragment after electrophoresis in 15% agarose gel is excised from the gel and transferred to 30 mL of NT buffer, dissolved at 50° C. for 5 to 10 minutes, and applied to a NucleoSpinExtractII column. Wash with NT 3 buffer and elute with 7.5 μL of NE buffer.

Oligonucleotide sequences SEQ ID NO 8—SEQ ID NO 145 encoding the hybrid protein SEQ ID NO 2 are obtained by complete nucleotide synthesis in several variants, where the sequence X₁X₂YX₃ was: AKY, RKY, NKY, DKY, CKY, QKY, EKY, GKY, HKY, IKY, LKY, KKY, MKY, FKY, RKY, PKY, OKY, SKY, UKY, TKY, WKY, YKY, VKY, ANY, RNY, NNY, DNY, CNY, QNY, ENY, GNY, HNY, INY, LNY, KNY, MNY, FNY, RNY, PNY, ONY, SNY, UNY, TNY, WNY, YNY, VNY, AKYH, RKYH, NKYH, DKYH, CKYH, QKYH, EKYH, GKYH, HKYH, IKYH, LKYH, KKYH, MKYH, FKYH, RKYH, PKYH, OKYH, SKYH, UKYH, TKYH, WKYH, YKYH, VKYH, ANYH, RNYH, NNYH, DNYH, CNYH, QNYH, ENYH, GNYH, HNYH, INYH, LNYH, KNYH, MNYH, FNYH, RNYH, PNYH, ONYH, SNYH, UNYH, TNYH, WNYH, YNYH, VNYH, AKYQ, RKYQ, NKYQ, DKYQ, CKYQ, QKYQ, EKYQ, GKYQ, HKYQ, IKYQ, LKYQ, KKYQ, MKYQ, FKYQ, RKYQ, PKYQ, OKYQ, SKYQ, UKYQ, TKYQ, WKYQ, YKYQ, VKYQ, ANYQ, RNYQ, NNYQ, DNYQ, CNYQ, QNYQ, ENYQ, GNYQ, HNYQ, INY, LNYQ, KNYQ, MNYQ, FNYQ, RNYQ, PNYQ, ONYQ, SNYQ, UNYQ, TNYQ, WNYQ, YNYQ or VNYQ.

In this case, the following variants of the hybrid protein were obtained, having the amino acid sequences of SEQ ID NO 146-SEQ ID NO 283

The resulting variants of the synthetic sequences SEQ ID NO 8-SEQ ID NO 145 are separately processed in 40 μL of buffer 20 mM Tris-acetate, 10 mM magnesium acetate, 50 mM potassium acetate, 100 μg/mL BSA restrictase XhoI (10 active units) and then in 40 μL of buffer 100 mM NaCl, 50 mM Tris-HCl, 10 mM MgCl₂, 100 μg/mL BSA restrictase NdeI (10 active units) for 1 hour at 37° C. Synthetic fragments after electrophoresis in 15% agarose gel are excised from the gel and transferred to 200 μL of NT buffer, dissolved at 50° C. for 5 to 10 minutes, and applied to a NucleoSpinExtractII column. Wash with NT 3 buffer and elute with 50 μL of NE buffer.

All the synthetic fragments obtained above were added separately in an amount of 2 μmol to solutions of 1 μg obtained from the DNA of the plasmid pET23b, the vector fragment described above in 10 μL of buffer (20 mM Tris-HCl, pH 7.56, 10 mM MgCl₂, 0.2 mM rATP, 10 mM dithiothreitol) and ligated with 10 active units. T4 DNA ligase for 12 hours at 10° C.

Tubes containing competent cells (XL1-Blue, Eurogen) are placed on ice and thawed completely, one tube per transformation. The cell suspension is gently mixed with a delicate shaking motion. An aliquot of the reaction mixture obtained after treatment with T4-DNA ligase is added to each tube and gently mixed with a delicate shaking motion. Tubes are incubated on ice for 20 to 30 minutes. The test tubes are transferred to a water bath (42° C.) for 30 to 45 seconds. The tubes are quickly transferred from the water bath to ice and incubated for 3 to 5 minutes. At least three volumes of SOB or SOC (Becton Dickinson) medium preheated to 37 to 42° C. are added, mixed, and incubated at 37° C. for 40 to 60 minutes in an orbital shaker incubator (Multitron, Infors) at 225 to 250 rpm. Sow the contents of the test tubes on Petri dishes with a diameter of 60 mm (Perint).

Aliquots of the resulting plasmid DNA are used to transform competent E. coli BL21 (DE3) cells. Transformants are plated on plates with LB agar medium, to which ampicillin is added to a final ampicillin concentration of 50 μg/mL. Plasmid DNA pET23b-CBD-HS-ES-GLP1 is isolated from the clones. Screening of recombinants is carried out using sequencing.

### Example 2 Preparation of E. coli BL21 (DE3)/CBD-HS-ES-GLP1 Producer Strain and Characterization of its Productivity

The E. coli BL21 (DE3)/CBD-HS-ES-GLP1 producer strain is obtained by transforming competent E. coli BL21 (DE3) cells with the plasmid, the preparation of which is described in Example 1.

The E. coli BL21 (DE3)/CBD-HS-ES-GLP1 producer strain is cultivated at 37° C. in 100 mL of LB liquid nutrient medium supplemented with ampicillin to a final ampicillin concentration of 100 μg/mL for 3 hours in Erlenmeyer flasks (1 L, Corning) in an orbital shaker-incubator with a rotation speed of 220 rpm until the optical density of the culture liquid at a wavelength of 600 nm is 0.7 to 0.8 units. Then, the biosynthesis of the recombinant protein is induced by adding isopropyl-β-D-1-thiogalactopyranoside to a final concentration of isopropyl-β-D-1-thiogalactopyranoside of 0.5 mM and incubated for 4 hours. Every hour a sample of 2 mL is taken, and the amount of culture corresponding to 1 mL is centrifuged for 10 minutes at 6000 rpm. The precipitated cells are transferred into 100 μL of lysis buffer with bromophenol blue dye, with the addition of 2-mercaptoethanol, incubated for 5 minutes at 98° C., 3 μL aliquots are used for electrophoresis in 14% SDS-PAGE. The gel is stained with 0.1% Coomassie R-250 and scanned with a Shimadzu CS-930 densitometer. The results are presented in FIG. 4. 4—cell lysate, 5—molecular weight standards. According to the results of densitometric analysis, the yield of the GLP-1 polypeptide is 35% relative to the total cell protein.

### Example 3. Preparation of GLP-1 Polypeptide

After the end of cultivation, according to Example 2, the cells of the producer of the recombinant protein (biomass) are separated by centrifugation (5000 g, 20 minutes, 4° C.), destroyed on an ultrasonic disintegrator (Elma) in a buffer solution (50 mM Tris/HCl, 10 mM EDTA, pH 8). Ammonium suphate is added to a final concentration of 200 g and incubated for 1 to 2 h, and inclusion bodies are separated by centrifugation (15000 g, 45 min). The inclusion bodies are extracted in buffer (50 mM Tris pH 11 8M Urea). The solubilized protein is applied to a metal chelate sorbent (Profinity IMAC, Ni-charged, Bio-Rad), and the protein is eluted with a buffer step of 0.250 M imidazole, 0.025 mM Tris, 2 M urea, 0.1 M NaCl, pH 8.

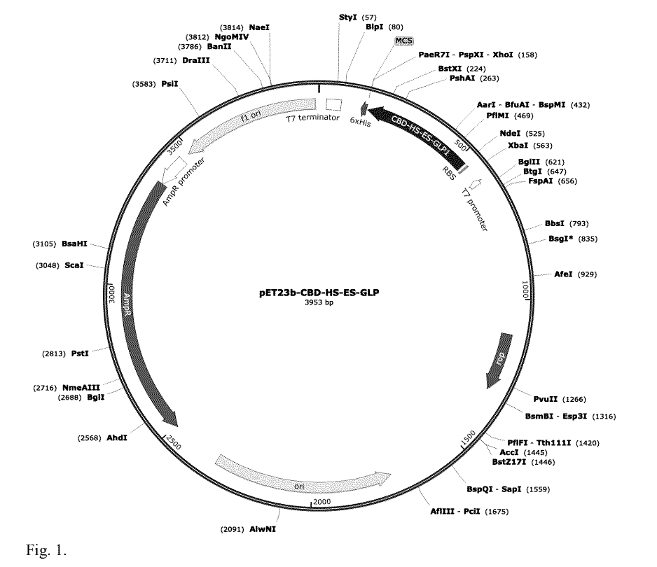

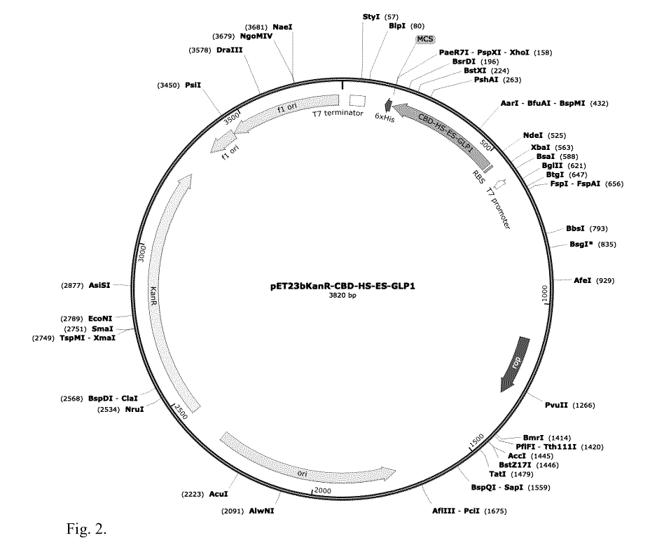

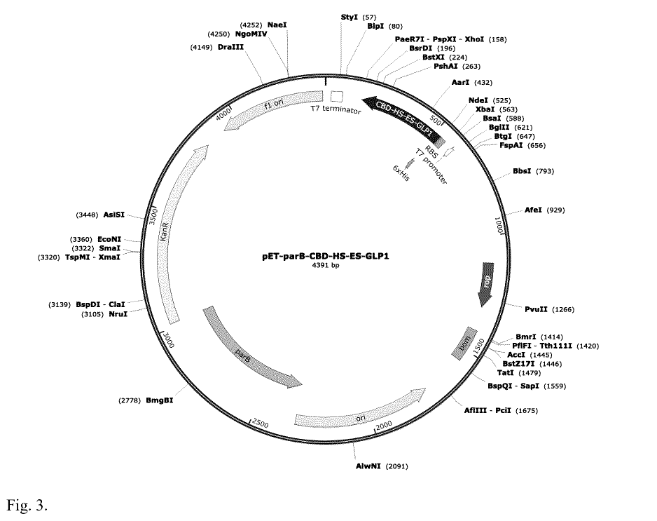

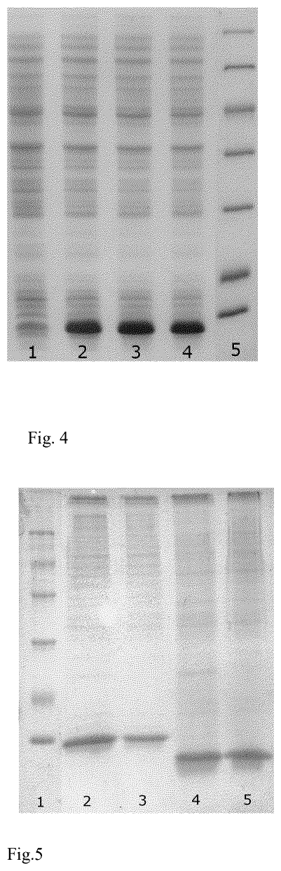

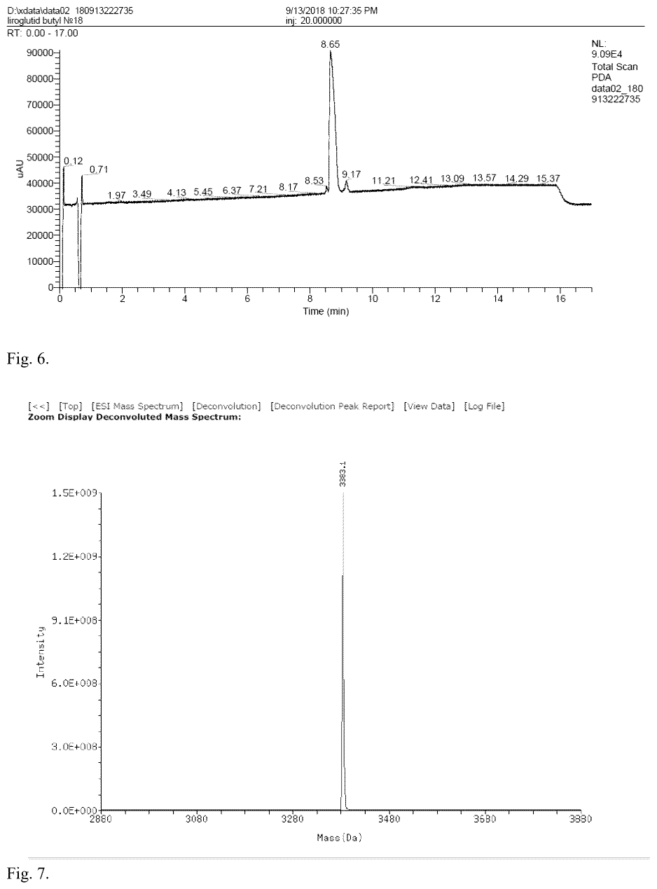

The eluate is concentrated on a 3 kDa ultrafiltration membrane, and enteropeptidase is added based on the ratio of 1 enzyme unit per 1 mg of the hybrid protein, thereby initiating the hybrid protein cleavage, and incubated at 22° C. for 14 to 19 hours. From the resulting mixture, 30 μL are sampled and incubated with bromophenol blue dye for 3 minutes at 100° C. 4-μL samples are used for electrophoresis in 15% SDS-PAGE. The gel was stained with Coomassie R-250 according to the standard technique and scanned with a Shimadzu CS-930 densitometer. The results are presented in FIG. 5. 1—molecular weight standards, 2 to 3—before incubation, and 4 to 5—after incubation. After incubation at 22° C. for 1 3 to 19 hours, adjust the pH of the solution with hydrochloric acid to 3.0 and centrifuge. Further purification and analysis of the GLP-1 polypeptide is carried out by reverse phase chromatography. The resulting GLP-1 polypeptide was identified by HPLC-MS. Chromatographic separation was carried out on a Phenomenex Aeris PEPTIDE 1.7u XB-C18150*2.1 mm column using an Accela UPLC (Thermo) chromatographic system in the gradient elution mode (linear gradient of acetonitrile concentration 5% to 55%). For mass spectrometric analysis, we used an LCQ Deca XP Plus (Thermo Finnigan) coupled ion trap detector in the electrospray ionization mode. Detection was performed using positive ion registration mode in a mass range of 200 to 2000 Da. FIGS. 6 and 7 show the result of a chromato-mass spectrometric analysis of a GLP-1 polypeptide. The purity of the preparation is 99%, according to the chromatogram in FIG. 6.

### Example 4 Construction of an Expression Plasmid with a Kanamycin Resistance Gene

The DNA variants of plasmid pET23b-CBD-HS-ES-GLP1 obtained in example 1 in the amount of 3 μg (1 μmol) are individually treated in 40 μL of buffer 20 mM Tris-acetate, 10 mM magnesium acetate, 50 mM potassium acetate, 100 μg/mL BSA restrictase PsiI (10 active units), and then in 40 μL buffer 100 mM NaCl, 50 mM Tris-HCl, 10 mM MgCl₂, 100 μg/mL BSA restrictase PciI (10 active units) for 1 hour at 37° C. After electrophoresis in 15% agarose gel, the vector fragment is excised from the gel, transferred to 200 μL of NT buffer, dissolved at 50° C. for 5 to 10 minutes, and applied to a NucleoSpinExtractII column. Wash with NT 3 buffer and elute with 50 μL of NE buffer.

DNA of plasmid pET28a (3 μg, 1 μmol) is treated in 6 mL of buffer 20 mM Tris-acetate, 10 mM magnesium acetate, 50 mM acetate to potassium, 100 μg/mL BSA restrictase PsiI (1000 active units), and then in 40 μL buffer 100 mM NaCl, 50 mM Tris-HCl, 10 mM MgCl₂, 100 μg/mL BSA restrictase PciI (1000 active units) for 1 hour at 37° C. After electrophoresis in 15% agarose gel, the vector fragment is excised from the gel, transferred to 3 mL of NT buffer, dissolved at 50° C. for 5 to 10 minutes, and applied to a NucleoSpinExtractII column. Wash with NT 3 buffer and elute with 7.5 mL of NE buffer.

The above-described fragment of plasmid pET28a in the amount of 2 μmol is added separately to solutions of 1 μg obtained from the DNA of plasmid pET23b-CBD-HS-ES-GLP1, the vector fragments described above in 10 μL of buffer (20 mM Tris-HCl, pH 7.56, 10 mM MgCl₂, 0.2 mM rATP, 10 mM dithiothreitol) and ligated with 10 active units. T4 DNA ligase for 12 hours at 10° C.

Tubes containing competent cells (XL1-Blue, Eurogen) are placed on ice and thawed completely, one tube per transformation. The cell suspension is gently mixed with a delicate shaking motion. An aliquot of the reaction mixture obtained after treatment with T4-DNA ligase is added to each tube and gently mixed with a delicate shaking motion. Tubes are incubated on ice for 20 to 30 minutes. The test tubes are transferred to a water bath (42° C.) for 30 to 45 seconds. The tubes are quickly transferred from the water bath to ice and incubated for 3 to 5 minutes. At least three volumes of SOB or SOC (Becton Dickinson) medium preheated to 37 to 42° C. are added, mixed, and incubated at 37° C. for 40 to 60 minutes in an orbital shaker incubator (Multitron, Infors) at 225 to 250 rpm. The contents of the test tubes are cultivated on Petri 60-mm dishes (Perint).

An aliquot of the resulting plasmid DNA is used to transform competent E. coli BL21 (DE3) cells. Transformants are plated on plates with LB agar medium, to which ampicillin is added to a final ampicillin concentration of 50 μg/mL. Clones isolated DNA plasmid pET23bKanR-CBD-HS-ES-GLP1. Screening of recombinants is carried out using sequencing.

### Example 5 Preparation of E. coli BL21 (DE3)/KanR/CBD-HS-ES-GLP1 Producer Strain and Characterization of its Productivity

The E. coli BL21 (DE3)/KanR/CBD-HS-ES-GLP1 producer strain is obtained by transforming competent E. coli BL21 (DE3) cells with the plasmid, the preparation of which is described in Example 4.

Producer strain E. coli BL21 (DE3)/KanR/CBD-HS-ES-GLP1 is cultivated at 37° C. in 100 mL of LB liquid nutrient medium supplemented with kanamycin sulphate to a final concentration of 50 μg/mL kanamycin sulphate supplemented for 3 hours in Erlenmeyer flasks (1 L, Corning) in an orbital shaker-incubator with a rotation speed of 220 rpm until the optical density of the culture liquid at a wavelength of 600 nm is 0.7 to 0.8 units. Then, the biosynthesis of the recombinant protein is induced by adding isopropyl-β-D-1-thiogalactopyranoside to a final concentration of isopropyl-β-D-1-thiogalactopyranoside of 0.5 mM and incubated for 4 hours. Every hour a sample of 2 mL is taken, and the amount of culture corresponding to 1 mL is centrifuged for 10 minutes at 6000 rpm. The precipitated cells are transferred into 100 μL of lysis buffer with bromophenol blue dye, with the addition of 2-mercaptoethanol, incubated for 5 minutes at 98° C., 3 μL aliquots are used for electrophoresis in 14% SDS-PAGE. The gel is stained with 0.1% Coomassie R-250 and scanned with a Shimadzu CS-930 densitometer. The results are presented in FIG. 8. 4—cell lysate, 5—molecular weight standards. According to the results of the densitometric analysis, the yield of the GLP-1 polypeptide is 35% relative to the total cell protein.

### Example 6 Construction of Expression Plasmid with a Genetic Construct Stability Enhancement Module

The DNA variants of plasmid pET23b-KanR-CBD-HS-ES-GLP1 obtained in example 4 in the amount of 3 μg (1 μmol) are individually treated in 40 μl of 20 mM buffer Tris-acetate, 10 mM magnesium acetate, 50 mM potassium acetate, 100 μg/ml BSA restrictase XmaI (10 active units), and then in 40 μl of 100 mM buffer NaCl, 50 mM Tris-HCl, 10 mM MgCl₂, 100 μg/ml BSA restriction enzyme PciI (10 active units) for 1 hour at 37° C. The vector fragment after electrophoresis in 15% agarose gel is excised from the gel and transferred to 200 μl of NT buffer, dissolved at 50° C. for 5-10 min, and applied to a NucleoSpinExtractII column. Wash with NT 3 buffer and elute with 50 μl of NE buffer.

The synthetic sequence of SEQ ID NO 7 (60 μmol) is treated in 6 ml of 20 mM buffer Tris-acetate, 10 mM magnesium acetate, 50 mM potassium acetate, 100 μg/ml BSA restrictase XmaI (1000 active units), and then in 6 ml of 100 mM buffer NaCl, 50 mM Tris-HCl, 10 mM MgCl₂, 100 μg/ml BSA restriction enzyme PciI (1000 units) for 1 hour at 37° C. The vector fragment after electrophoresis in 15% agarose gel is excised from the gel and transferred to 3 ml of NT buffer, dissolved at 50° C. for 5-10 min, and applied to a NucleoSpinExtractII column. Wash with NT 3 buffer and elute with 50 μl of NE buffer. The obtained fragment of the synthetic sequence SEQ ID NO 145 described above in the amount of 2 μmol is added separately to 1 μg solutions obtained from the DNA of the pET23b-KanR-CBD-HS-ES-GLP1 plasmid, the vector fragments described above in 10 μl of buffer (20 mm tris-HCl, pH 7.56, 10 mM MgCl₂, 0.2 mM rATP, 10 mM dithiothreitol) and ligated with 10 units T4 DNA ligase for 12 hours at 10° C.

Tubes with competent cells (XL1-Blue, Eurogen) are placed on ice until the contents are completely thawed at the rate of one tube per transformation. Gently mix the cell suspension with light shaking. Add an aliquots of the reaction mixture obtained after treatment with T4-DNA ligase to each tube, gently mix the contents with gentle shaking. Incubate tubes on ice for 20-30 minutes. Transfer the test tubes to a water bath (42° C.) for 30-45 sec. Quickly transfer the tubes from the water bath to ice and incubate for 3-5 minutes. Add at least 3 volumes of preheated up to 37-42° C. SOB or SOC medium (Becton Dickinson), mix the contents and incubate at 37° C. for 40-60 min in an orbital shaker-incubator (Multitron, Infors) at a speed of 225-250 rpm. Sow the contents of the test tubes on Petri dishes with a diameter of 60 mm (Perint).

An aliquot of the resulting plasmid DNA is used to transform competent E. coli BL21 (DE3) cells. Transformants are plated on plates with LB agar medium, to which ampicillin is added to a final ampicillin concentration of 50 μg/ml. Clones isolated DNA plasmid pET-parB-CBD-HS-ES-GLP1. Screening for recombinants is done by sequencing.

### Example 7. Preparation of the Producer Strain E. coli BL21 (DE 3)/parB-CBD-HS-ES-GLP1 and Characterization of its Productivity

The producer strain E. coli BL21 (DE 3)/parB-CBD-HS-ES-GLP1 is obtained by transforming competent cells of E. coli BL21 (DE3) with the plasmid, the preparation of which is described in example 6.

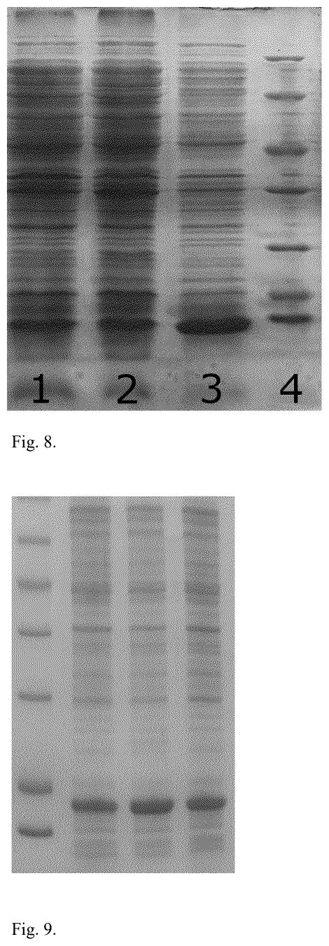

Producer strain E. coli BL21 (DE3)/parB-CBD-HS-ES-GLP1 is cultivated at 37° C. in 100 ml of LB liquid nutrient medium supplemented with kanamycin sulphate to a final concentration of kanamycin sulphate 50 μg/ml for 3 h in Erlenmeyer flasks (1 1, Corning) in an orbital shaker-incubator with a rotation speed of 220 rpm until the optical density of the culture liquid at a wavelength of 600 nm is 0.7-0.8 units. Then the biosynthesis of the recombinant protein is induced by adding isopropyl-β-D-1-thiogalactoprianoside to a final concentration of iso-propyl-β-D-1-thiogalactoprianoside of 0.5 mM and incubated for 4 hours. A sample of 2 ml is taken every hour., the amount of culture corresponding to 1 ml, centrifuged for 10 min at 6000 rpm. The precipitated cells are transferred into 100 μl lysing buffer with bromophenol blue dye, with the addition of 2-mercaptoethonol, incubated for 5 min at 98° C., aliquots of 3 μl are used for electrophoresis in 14% SDS-PAGE. The gel is stained with a 0.1% Coomassie R-250 solution and scanned using a Shimadzu CS-930 densitometer. The results are presented in FIG. 9. 4—cell lysate, 5—molecular weight standards. The yield of the GLP-1 polypeptide, according to the results of densitometric analysis, is 40% relative to the total cell protein.

### Example 8 Preparation of the Producer Strain E. coli BL21 (DE3)/KanR/CBD-HS-ES-GLP1

The BL21 (DE3)/KanR-CBD-HS-ES-GLP1 strain is prepared by transforming Escherichia coli BL21 (DE3) cells with the 3816 bp pET23bKanR-CBD-HS-ES-GLP1 expression plasmid encoding the hybrid protein SEQ ID NO 2, consisting of the following key genetic elements:
a)-kanamycin resistance gene (KanR) and bacterial kanamycin resistance gene promoter;
b) origin of bacteriophage f1 replication;
c) T7 promoter and lacO operator;
d)-A synthetic nucleotide sequence is shown in SEQ ID NO 5 encoding a CBD-HS-ES-GLP1 hybrid protein (SEQ ID NO 2) containing a GLP-1 polypeptide (SEQ ID NO 1), an enterokinase recognition site of SEQ ID NO 4, a leader a polypeptide (SEQ ID NO 3) having an HS hexahistidine site and a CBD chitin-binding site.

According to the invention, the starting material for creating a producer strain is a strain of E. coli BL21 (DE3) known from the prior art. Expression plasmid 3816 bp long, consisting of key genetic elements (a) to (d), located relative to each other as shown in FIG. 2, transform cells of E. coli strain BL21 (DE3).

Preferably (without limitation), for the introduction of the specified plasmid in the cells of the strain E. coli BL21 (DE3) use the method of electroporation, known from the prior art and included in the present description by reference. The skilled specialist will appreciate that other embodiments of the present invention may use other transformation methods known in the art to introduce the plasmid into E. coli BL21 (DE3) cells, e. g., polyethylene glycol method and calcium chloride method. Moreover, for the present invention, for introduction into E. coli BL21 (DE3) cells, the plasmid shown in FIG. 2, and other transformation methods not explicitly mentioned in the present description, which are currently known in the prior art or will be created subsequently, can be used. In implementing specific embodiments of this invention, a specialist can choose the most optimal method for cell transformation based on their existing knowledge.

Transformed cells are plated on Petri dishes with agar medium with the addition of the selection agent kanamycin sulphate to a final concentration of kanamycin sulphate of 50 μg/mL of the cell. From clones resistant to kanamycin, the pET23bKanR-CBD-HS-ES-GLP1 plasmid DNA was isolated and analyzed by sequencing.

The proposed strain-producer Escherichia coli BL21 (DE3)/KanR-CBD-HS-ES-GLP1 is characterized by the following features:

Morphological features: rod-shaped cells, gram-negative, non-spore-bearing.

Cultural characteristics: cells grow well on simple nutrient media. When growing on agar medium “LB” (per 1 1 10 g peptone, 5 g yeast extract, 10 g NaCl, 20 g agar)—the colonies are round, smooth, cloudy, shiny, and gray, and the edges are even.

Physical and biological signs: cells grow at 4° C. to 40° C. with an optimal pH value of 6.8 to 7.5. As a source of nitrogen, both mineral salts in the ammonium form and organic compounds, including peptone, tryptone, yeast extract, amino acids, etc., are used. Amino acids, glycerol, and carbohydrates are used as a carbon source.

Antibiotic resistance: cells show resistance to kanamycin (up to 500 μg/mL).

Preparation of the producer strain E. coli BL21 (DE3)/KanR-parB-CBD-HS-ES-GLP1

E. coli BL21 (DE3)/KanR-parB-CBD-HS-ES-GLP1 is prepared by transforming Escherichia coli BL21 (DE3) cells with the expression plasmid pET-KanR-parB-CBD-HS-ES-GLP1 4391 bp in size, encoding a hybrid protein SEQ ID NO 2, consisting of the following key genetic elements:
a) a kanamycin resistance gene (KanR) and a bacterial kanamycin resistance gene promoter;
b) origin of bacteriophage fl replication;
c) T7 promoter and lacO operator;
d) parB—hok/sok locus;
e) A synthetic nucleotide sequence is shown in SEQ ID NO 5 encoding a CBD-HS-ES-GLP1 hybrid protein (SEQ ID NO 2) containing a GLP-1 polypeptide (SEQ ID NO 1), an enterokinase recognition site of SEQ ID NO 4, a leader polypeptide (SEQ ID NO 3) containing an HS hexahistidine site and a CBD chitin-binding site.

According to the invention, the starting material for creating a producer strain is a strain of E. coli BL21 (DE3) known from the prior art. Expression plasmid 4387 bp long, consisting of key genetic elements (a) to (e), located relative to each other as shown in FIG. 3, transform cells of E. coli strain BL21 (DE3).

Preferably (without limitation), for the introduction of the specified plasmid in the cells of the strain E. coli BL21 (DE3) use the method of electroporation, known from the prior art and included in the present description by reference. The skilled specialist will appreciate that other embodiments of the present invention may use other transformation methods known in the art to introduce the plasmid into E. coli BL21 (DE3) cells, e. g., polyethylene glycol method and calcium chloride method. Also for the present invention, for introduction into E. coli BL21 (DE3) cells, the plasmid shown in FIG. 3, and other transformation methods not explicitly mentioned in the present description, which are currently known in the prior art or will be created subsequently, can be used. In implementing specific embodiments of this invention, a specialist can choose the most optimal method for cell transformation based on their existing knowledge.

Transformed cells are plated on Petri dishes with agar medium supplemented with selection agent kanamycin sulphate to a final concentration of kanamycin sulphate of 50 μg/mL of the cell. From the kanamycin-resistant clones, pET-parB-CBD-HS-ES-GLP1 plasmid DNA was isolated and analyzed by sequencing.

The proposed strain-producer E. coli BL21 (DE3)/KanR-parB-CBD-HS-ES-GLP1 is characterized by the following features:

Morphological features: rod-shaped cells, gram-negative, non-spore-bearing.

Cultural characteristics: cells grow well on simple nutrient media. When growing on agar medium “LB” (per 1 L-10 g peptone, 5 g yeast extract, 10 g NaCl, 20 g agar)—the colonies are round, smooth, cloudy, shiny, and gray, and the edges are even.

Physical and biological signs: cells grow at 4° C. to 40° C. with an optimal pH value of 6.8 to 7.5. As a source of nitrogen, both mineral salts in the ammonium form and organic compounds, including peptone, tryptone, yeast extract, amino acids, etc., are used. Amino acids, glycerol, and carbohydrates are used as a carbon source.

Antibiotic resistance: cells show resistance to kanamycin (up to 500 g/mL).

1. A hybrid protein CBD-HS-ES-GLP1 for producing a GLP-1 polypeptide, having the amino acid sequence of SEQ ID NO 2, containing the amino acid sequence X₁X₂YX₃, chitin-binding domain (CBD), enteropeptidase specific recognition site(ES), a hexahistidine domain (HS), a GLP-1 polypeptide having the amino acid sequence of SEQ ID NO 1, where X₁X₂YX₃ is an amino acid sequence in which X₁ is selected from the group consisting of A, R, N, D, C, Q, E, G, H, I, L, K, M, F, R, P, O, S, U, T, W, Y, and V, X₂ is selected from K or N, and X₃ missing or selected from H or Q.

2. The hybrid protein CBD-HS-ES-GLP1 of claim 1, characterized in that the amino acid sequence X₁X₂YX₃ is VKY.

3. The hybrid protein CBD-HS-ES-GLP1 of claim 1, characterized in that the amino acid sequence X₁X₂YX₃ is VNY.

4. A recombinant plasmid DNA pET23b-CBD-HS-ES-GLP1 for the expression of the hybrid protein of claim 1, which consists of the following key genetic elements:
T7 promoter and lacO operator;
synthetic nucleotide sequence presented in SEQ ID NO 5, encoding the hybrid protein of claim 1;
antibiotic resistance gene for selection of recombinant cells;
origin of DNA replication of bacteriophage f1 (f1 ori).

5. The recombinant plasmid DNA pET23b-CBD-HS-ES-GLP1 of claim 4, characterized in that it has a length of 3953 bp, contains the ampicillin resistance gene (AmpR) as an antibiotic resistance gene and the bacterial promoter of the ampicillin resistance gene (AmpR promoter), and has an arrangement of elements as shown in FIG. 1.

6. The recombinant plasmid DNA pET23b-CBD-HS-ES-GLP1 of claim 4, characterized in that it has a length of 3820 bp, contains the antibiotic resistance gene kanamycin (KanR) as an antibiotic resistance gene, and has an arrangement of elements as shown in FIG. 2.

7. The recombinant plasmid DNA pET23b-CBD-HS-ES-GLP1 of claim 4, characterized in that it has a length of 4391 bp, contains the antibiotic resistance gene kanamycin (KanR) as an antibiotic resistance gene, additionally contains a hok/sok plasmid stabilizing locus, and has an arrangement of elements as shown in FIG. 3.

8. A Escherichia coli BL21 (DE3)/CBD-HS-ES-GLP1 strain which produces the hybrid protein and contains the recombinant plasmid DNA pET23b-CBD-HS-ES-GLP1 of claim 4.

9. The Escherichia coli strain of claim 8, characterized in that it was obtained by transforming cells of the Escherichia coli B 21 (DE3) strain with recombinant plasmid DNA pET23b-CBD-HS-ES-GLP1 of claim 5.

10. The Escherichia coli strain of claim 8, characterized in that it was obtained by transforming the Escherichia coli BL21 (DE3) strain cells with the recombinant plasmid DNA pET23b-CBD-HS-ES-GLP1 of claim 6.

11. The Escherichia coli strain of claim 8, characterized in that it was obtained by transforming the Escherichia coli BL21 (DE3) strain cells with the recombinant plasmid DNA pET23b-CBD-HS-ES-GLP1 of claim 7.

12. A method for producing a GLP-1 polypeptide having the amino acid sequence of SEQ ID NO 1, comprising the stages:
a) cultivating the Escherichia coli BL21 (DE3)/CBD-HS-ES-GLP1 producer strain of any one of claims 8-11 to prepare cell biomass of the producer strain, which produces the hybrid protein CBD-HS-ES-GLP1 of claim 1;
b) isolating the hybrid protein CBD-HS-ES-GLP1 of claim 1 from the cell biomass of the producer strain obtained at stage (a);
c) enzymatic cleavage of the hybrid protein CBD-HS-ES-GLP1 obtained at stage (b) to form a GLP-1 polypeptide with the amino acid sequence of SEQ ID NO 1;
d) purification of the polypeptide GLP-1 obtained at stage (c).

13. The method of claim 12, characterized in that, at stage (b), the cells are separated from the culture fluid, the cells are disintegrated, the inclusion bodies are isolated from the resulting disintegrate, and the inclusion bodies are solubilized.

14. The method of claim 12 or 13, characterized in that, at stage (b) when separating the inclusion bodies from the resulting disintegrate, ammonium sulphate is added

15. The method of any one of claims 12-14, characterized in that, at stage (a), the cultivation of Escherichia coli BL21 (DE3)/CBD-HS-ES-GLP1 cells is carried out in a growth medium for at least 7 hours.

16. The method of any one of claims 12-15, characterized in that, at stage (a), the biosynthesis of the recombinant protein is induced by Escherichia coli BL21 (DE3)/CBD-HS-ES-GLP1 cells 3 hours after the start of cultivation using isopropyl-β-D-1-thiogalactopyranoside.

17. The method of any one of claims 12-16, characterized in that, at stage (c), the splitting of the hybrid protein is carried out by enteropeptidase.

18. The method of any one of claims 12-17, characterized in that, at stage (b), the purification of the GLP-1 polypeptide is carried out using one chromatography stage.

19. The method of any one of claims 12-17, characterized in that, at stage (b), the purification of the GLP-1 polypeptide is carried out using several chromatography stages.

20. The method of any one of claims 12-19, characterized in that, at stage (b), the purification of the hybrid protein CBD-HS-ES-GLP1 is carried out chromatographically on a metal-chelate sorbent.

21. The method of any one of claims 12-19, characterized in that, at stage (b), the purification of the hybrid protein CBD-HS-ES-GLP1 is carried out chromatographically on a cation-exchange sorbent.

22. The method of any one of claims 12-19, characterized in that, at stage (b), the purification of the hybrid protein CBD-HS-ES-GLP1 is carried out chromatographically on a chitin sorbent.

23. The method of any one of claims 12-21, characterized in that, at stage (d), the purification of the polypeptide GLP-1 is carried out using reverse-phase chromatography.

24. The method of any one of claims 12-22, characterized in that, at stage (d), the purification of the polypeptide GLP-1 is carried out using size-exclusion chromatography.

25. The method of any one of claims 12-22, characterized in that, at stage (d), the purification of the polypeptide GLP-1 is carried out by precipitation with ammonium sulphate and extraction of impurities with a buffer solution.

26. The method of any one of claims 12-22, characterized in that, at stage (d), the purification of the hybrid protein CBD-HS-ES-GLP1 is carried out chromatographically on a chitin sorbent.


SEQUENCE LISTING (283 sequences)

SEQ ID NO: 1 - protein, 31 aa, unidentified
HAEGTFTSDVSSYLEGQAAKEFIAWLVRGRG

SEQ ID NO: 2 - protein, 121 aa, synthetic construct
MXXYXHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 3 - protein, 68 aa, synthetic construct
GASMTGGQQMGSLTTNPGVSAWQVNTAYTAGQLVTYNGRTYRSLQPHTSLAGWEPSNVPALWQLQSLG

SEQ ID NO: 4 - protein, 5 aa, synthetic construct
DDDDR

SEQ ID NO: 5 - RNA, 351 nt, synthetic construct
  source 1..351: mol_type = other RNA
catcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 6 - protein, 5 aa, synthetic construct
DDDDK

SEQ ID NO: 7 - RNA, 1651 nt, synthetic construct
  source 1..1651: mol_type = other RNA
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgacatcagcaaggagaaaggggctaccggcgaaccagcagcccctttataaaggcgcttcagtagtcagaccagcatcagtcctgaaaaggcgggcctgcgcccgcctccaggttgctacttaccggattcgtaagccatgaaagccgccacctccctgtgtccgtctctgtaacgaatctcgcacagcgattttcgtgtcagataagtgaatatcaacagtgtgagacacacgatcaacacacaccagacaagggaacttcgtggtagtttcatggccttcttctccttgcgcaaagcgcggtaagaggctatcctgatgtggactagacatagggatgcctcgtggtggttaatgaaaattaacttactacggggctatcttctttctgccacacaacacggcaacaaaccaccttcacgtcatgaggcagaaagcctcaagcgccgggcacatcatagcccatatacctgcacgctgaccacactcactttccctgaaaataatccgctcattcagaccgttcacgggaaatccgtgtgattgttgccgcatcacgctgcctcccggagtttgttggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggg

SEQ ID NO: 8 - RNA, 363 nt, synthetic construct
  source 1..363: mol_type = other RNA
atggcgaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 9 - RNA, 363 nt, synthetic construct
  source 1..363: mol_type = other RNA
atgagaaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 10 - RNA, 363 nt, synthetic construct
  source 1..363: mol_type = other RNA
atgaataaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 11 - RNA, 363 nt, synthetic construct
  source 1..363: mol_type = other RNA
atggataaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 12 - RNA, 363 nt, synthetic construct
  source 1..363: mol_type = other RNA
atgtgcaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 13 - RNA, 363 nt, synthetic construct
  source 1..363: mol_type = other RNA
atgcagaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 14 - RNA, 363 nt, synthetic construct
  source 1..363: mol_type = other RNA
atggaaaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 15 - RNA, 363 nt, synthetic construct
  source 1..363: mol_type = other RNA
atgggtaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 16 - RNA, 363 nt, synthetic construct
  source 1..363: mol_type = other RNA
atgcataaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 17 - RNA, 363 nt, synthetic construct
  source 1..363: mol_type = other RNA
atgattaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 18 - RNA, 363 nt, synthetic construct
  source 1..363: mol_type = other RNA
atgctgaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 19 - RNA, 363 nt, synthetic construct
  source 1..363: mol_type = other RNA
atgaaaaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 20 - RNA, 363 nt, synthetic construct
  source 1..363: mol_type = other RNA
atgatgaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 21 - RNA, 363 nt, Synthetic construct
  source 1..363: mol_type = other RNA
atgtttaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 22 - RNA, 363 nt, Synthetic construct; identical to SEQ ID NO: 9
  source 1..363: mol_type = other RNA

SEQ ID NO: 23 - RNA, 363 nt, Synthetic construct
  source 1..363: mol_type = other RNA
atgccgaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 24 - RNA, 360 nt, Synthetic construct
  source 1..360: mol_type = other RNA
atgaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 25 - RNA, 363 nt, Synthetic construct
  source 1..363: mol_type = other RNA
atgagcaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 26 - RNA, 360 nt, Synthetic construct; identical to SEQ ID NO: 24
  source 1..360: mol_type = other RNA

SEQ ID NO: 27 - RNA, 363 nt, Synthetic construct
  source 1..363: mol_type = other RNA
atgaccaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 28 - RNA, 363 nt, Synthetic construct
  source 1..363: mol_type = other RNA
atgtggaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 29 - RNA, 363 nt, Synthetic construct
  source 1..363: mol_type = other RNA
atgtataaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 30 - RNA, 363 nt, Synthetic construct
  source 1..363: mol_type = other RNA
atggtgaaatatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 31 - RNA, 363 nt, Synthetic construct
  source 1..363: mol_type = other RNA
atggcgaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 32 - RNA, 363 nt, Synthetic construct
  source 1..363: mol_type = other RNA
atgagaaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 33 - RNA, 363 nt, Synthetic construct
  source 1..363: mol_type = other RNA
atgaataattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 34 - RNA, 363 nt, Synthetic construct
  source 1..363: mol_type = other RNA
atggataattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 35 - RNA, 363 nt, Synthetic construct
  source 1..363: mol_type = other RNA
atgtgcaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 36 - RNA, 363 nt, Synthetic construct
  source 1..363: mol_type = other RNA
atgcagaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 37 - RNA, 363 nt, Synthetic construct
  source 1..363: mol_type = other RNA
atggaaaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 38 - RNA, 363 nt, Synthetic construct
  source 1..363: mol_type = other RNA
atgggtaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 39 - RNA, 363 nt, Synthetic construct
  source 1..363: mol_type = other RNA
atgcataattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 40 - RNA, 363 nt, Synthetic construct
  source 1..363: mol_type = other RNA
atgattaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 41 - RNA, 363 nt, Synthetic construct
  source 1..363: mol_type = other RNA
atgctgaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 42 - RNA, 363 nt, Synthetic construct
  source 1..363: mol_type = other RNA
atgaaaaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 43 - RNA, 363 nt, Synthetic construct
  source 1..363: mol_type = other RNA
atgatgaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 44 - RNA, 363 nt, Synthetic construct
  source 1..363: mol_type = other RNA
atgtttaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 45 - RNA, 363 nt, Synthetic construct; identical to SEQ ID NO: 32
  source 1..363: mol_type = other RNA

SEQ ID NO: 46 - RNA, 363 nt, Synthetic construct
  source 1..363: mol_type = other RNA
atgccgaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 47 - RNA, 360 nt, Synthetic construct
  source 1..360: mol_type = other RNA
atgaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 48 - RNA, 363 nt, Synthetic construct
  source 1..363: mol_type = other RNA
atgagcaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 49 - RNA, 360 nt, Synthetic construct; identical to SEQ ID NO: 47
  source 1..360: mol_type = other RNA

SEQ ID NO: 50 - RNA, 363 nt, Synthetic construct
  source 1..363: mol_type = other RNA
atgaccaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 51 - RNA, 363 nt, Synthetic construct
  source 1..363: mol_type = other RNA
atgtggaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 52 - RNA, 363 nt, Synthetic construct
  source 1..363: mol_type = other RNA
atgtataattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 53 - RNA, 363 nt, Synthetic construct
  source 1..363: mol_type = other RNA
atggtgaattatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 54 - RNA, 366 nt, Synthetic construct
  source 1..366: mol_type = other RNA
atggcgaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 55 - RNA, 366 nt, Synthetic construct
  source 1..366: mol_type = other RNA
atgagaaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 56 - RNA, 366 nt, Synthetic construct
  source 1..366: mol_type = other RNA
atgaataaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 57 - RNA, 366 nt, Synthetic construct
  source 1..366: mol_type = other RNA
atggataaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 58 - RNA, 366 nt, Synthetic construct
  source 1..366: mol_type = other RNA
atgtgcaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 59 - RNA, 366 nt, Synthetic construct
  source 1..366: mol_type = other RNA
atgcagaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 60 - RNA, 366 nt, Synthetic construct
  source 1..366: mol_type = other RNA
atggaaaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 61 - RNA, 366 nt, Synthetic construct
  source 1..366: mol_type = other RNA
atgggtaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 62 - RNA, 366 nt, Synthetic construct
  source 1..366: mol_type = other RNA
atgcataaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 63 - RNA, 366 nt, Synthetic construct
  source 1..366: mol_type = other RNA
atgattaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 64 - RNA, 366 nt, Synthetic construct
  source 1..366: mol_type = other RNA
atgctgaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 65 - RNA, 366 nt, Synthetic construct
  source 1..366: mol_type = other RNA
atgaaaaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 66 - RNA, 366 nt, Synthetic construct
  source 1..366: mol_type = other RNA
atgatgaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 67 - RNA, 366 nt, Synthetic construct
  source 1..366: mol_type = other RNA
atgtttaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 68 - RNA, 366 nt, Synthetic construct; identical to SEQ ID NO: 55
  source 1..366: mol_type = other RNA

SEQ ID NO: 69 - RNA, 366 nt, Synthetic construct
  source 1..366: mol_type = other RNA
atgccgaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 70 - RNA, 363 nt, Synthetic construct
  source 1..363: mol_type = other RNA
atgaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 71 - RNA, 366 nt, Synthetic construct
  source 1..366: mol_type = other RNA
atgagcaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 72 - RNA, 363 nt, Synthetic construct; identical to SEQ ID NO: 70
  source 1..363: mol_type = other RNA

SEQ ID NO: 73 - RNA, 366 nt, Synthetic construct
  source 1..366: mol_type = other RNA
atgaccaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 74 - RNA, 366 nt, Synthetic construct
  source 1..366: mol_type = other RNA
atgtggaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 75 - RNA, 366 nt, Synthetic construct
  source 1..366: mol_type = other RNA
atgtataaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 76 - RNA, 366 nt, Synthetic construct
  source 1..366: mol_type = other RNA
atggtgaaatatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 77 - RNA, 366 nt, Synthetic construct
  source 1..366: mol_type = other RNA
atggcgaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 78 - RNA, 366 nt, Synthetic construct
  source 1..366: mol_type = other RNA
atgagaaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 79 - RNA, 366 nt, Synthetic construct
  source 1..366: mol_type = other RNA
atgaataattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 80 - RNA, 366 nt, Synthetic construct
  source 1..366: mol_type = other RNA
atggataattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 81 - RNA, 366 nt, Synthetic construct
  source 1..366: mol_type = other RNA
atgtgcaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 82 - RNA, 366 nt, Synthetic construct
  source 1..366: mol_type = other RNA
atgcagaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 83 - RNA, 366 nt, Synthetic construct
  source 1..366: mol_type = other RNA
atggaaaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 84 - RNA, 366 nt, Synthetic construct
  source 1..366: mol_type = other RNA
atgggtaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 85 - RNA, 366 nt, Synthetic construct
  source 1..366: mol_type = other RNA
atgcataattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 86 - RNA, 366 nt, Synthetic construct
  source 1..366: mol_type = other RNA
atgattaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 87 - RNA, 366 nt, Synthetic construct
  source 1..366: mol_type = other RNA
atgctgaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 88 - RNA, 366 nt, Synthetic construct
  source 1..366: mol_type = other RNA
atgaaaaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 89 - RNA, 366 nt, Synthetic construct
  source 1..366: mol_type = other RNA
atgatgaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 90 - RNA, 366 nt, Synthetic construct
  source 1..366: mol_type = other RNA
atgtttaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 91 - RNA, 366 nt, Synthetic construct; identical to SEQ ID NO: 78
  source 1..366: mol_type = other RNA

SEQ ID NO: 92 - RNA, 366 nt, Synthetic construct
  source 1..366: mol_type = other RNA
atgccgaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 93 - RNA, 363 nt, Synthetic construct
  source 1..363: mol_type = other RNA
atgaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 94 - RNA, 366 nt, Synthetic construct
  source 1..366: mol_type = other RNA
atgagcaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 95 - RNA, 363 nt, Synthetic construct; identical to SEQ ID NO: 93
  source 1..363: mol_type = other RNA

SEQ ID NO: 96 - RNA, 366 nt, Synthetic construct
  source 1..366: mol_type = other RNA
atgaccaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 97 - RNA, 366 nt, Synthetic construct
  source 1..366: mol_type = other RNA
atgtggaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 98 - RNA, 366 nt, Synthetic construct
  source 1..366: mol_type = other RNA
atgtataattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 99 - RNA, 366 nt, Synthetic construct
  source 1..366: mol_type = other RNA
atggtgaattatcatcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 100 - RNA, 366 nt, Synthetic construct
  source 1..366: mol_type = other RNA
atggcgaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 101 - RNA, 366 nt, Synthetic construct
  source 1..366: mol_type = other RNA
atgagaaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 102 - RNA, 366 nt, synthetic construct
  source 1..366: mol_type = other RNA
atgaataaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 103 - RNA, 366 nt, Synthetic construct
  source 1..366: mol_type = other RNA
atggataaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 104 - RNA, 366 nt, Synthetic construct
  source 1..366: mol_type = other RNA
atgtgcaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 105 - RNA, 366 nt, Synthetic construct
  source 1..366: mol_type = other RNA
atgcagaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 106 - RNA, 366 nt, Synthetic construct
  source 1..366: mol_type = other RNA
atggaaaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 107 - RNA, 366 nt, Synthetic construct
  source 1..366: mol_type = other RNA
atgggtaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 108 - RNA, 366 nt, Synthetic construct
  source 1..366: mol_type = other RNA
atgcataaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 109 - RNA, 366 nt, Synthetic construct
  source 1..366: mol_type = other RNA
atgattaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 110 - RNA, 366 nt, Synthetic construct
  source 1..366: mol_type = other RNA
atgctgaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 111 - RNA, 366 nt, Synthetic construct
  source 1..366: mol_type = other RNA
atgaaaaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 112 - RNA, 366 nt, Synthetic construct
  source 1..366: mol_type = other RNA
atgatgaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 113 - RNA, 366 nt, Synthetic construct
  source 1..366: mol_type = other RNA
atgtttaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 114 - RNA, 366 nt, Synthetic construct; identical to SEQ ID NO: 101
  source 1..366: mol_type = other RNA

SEQ ID NO: 115 - RNA, 366 nt, Synthetic construct
  source 1..366: mol_type = other RNA
atgccgaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 116 - RNA, 363 nt, Synthetic construct
  source 1..363: mol_type = other RNA
atgaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 117 - RNA, 366 nt, Synthetic construct
  source 1..366: mol_type = other RNA
atgagcaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 118 - RNA, 363 nt, Synthetic construct; identical to SEQ ID NO: 116
  source 1..363: mol_type = other RNA

SEQ ID NO: 119 - RNA, 366 nt, Synthetic construct
  source 1..366: mol_type = other RNA
atgaccaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 120 - RNA, 366 nt, Synthetic construct
  source 1..366: mol_type = other RNA
atgtggaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 121 - RNA, 366 nt, Synthetic construct
  source 1..366: mol_type = other RNA
atgtataaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 122 - RNA, 366 nt, Synthetic construct
  source 1..366: mol_type = other RNA
atggtgaaatatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 123 - RNA, 366 nt, Synthetic construct
  source 1..366: mol_type = other RNA
atggcgaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 124 - RNA, 366 nt, Synthetic construct
  source 1..366: mol_type = other RNA
atgagaaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 125 - RNA, 366 nt, Synthetic construct
  source 1..366: mol_type = other RNA
atgaataattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 126 - RNA, 366 nt, Synthetic construct
  source 1..366: mol_type = other RNA
atggataattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 127 - RNA, 366 nt, Synthetic construct
  source 1..366: mol_type = other RNA
atgtgcaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 128 - RNA, 366 nt, Synthetic construct
  source 1..366: mol_type = other RNA
atgcagaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 129 - RNA, 366 nt, Synthetic construct
  source 1..366: mol_type = other RNA
atggaaaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 130 - RNA, 366 nt, Synthetic construct
  source 1..366: mol_type = other RNA
atgggtaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 131 - RNA, 366 nt, Synthetic construct
  source 1..366: mol_type = other RNA
atgcataattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 132 - RNA, 363 nt, Synthetic construct; identical to SEQ ID NO: 40
  source 1..363: mol_type = other RNA

SEQ ID NO: 133 - RNA, 366 nt, Synthetic construct
  source 1..366: mol_type = other RNA
atgctgaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 134 - RNA, 366 nt, Synthetic construct
  source 1..366: mol_type = other RNA
atgaaaaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 135 - RNA, 366 nt, Synthetic construct
  source 1..366: mol_type = other RNA
atgatgaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 136 - RNA, 366 nt, Synthetic construct
  source 1..366: mol_type = other RNA
atgtttaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 137 - RNA, 366 nt, Synthetic construct; identical to SEQ ID NO: 124
  source 1..366: mol_type = other RNA

SEQ ID NO: 138 - RNA, 366 nt, Synthetic construct
  source 1..366: mol_type = other RNA
atgccgaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 139 - RNA, 363 nt, Synthetic construct
  source 1..363: mol_type = other RNA
atgaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 140 - RNA, 366 nt, Synthetic construct
  source 1..366: mol_type = other RNA
atgagcaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 141 - RNA, 363 nt, Synthetic construct; identical to SEQ ID NO: 139
  source 1..363: mol_type = other RNA

SEQ ID NO: 142 - RNA, 366 nt, Synthetic construct
  source 1..366: mol_type = other RNA
atgaccaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 143 - RNA, 366 nt, Synthetic construct
  source 1..366: mol_type = other RNA
atgtggaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 144 - RNA, 366 nt, Synthetic construct
  source 1..366: mol_type = other RNA
atgtataattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 145 - RNA, 366 nt, Synthetic construct
  source 1..366: mol_type = other RNA
atggtgaattatcagcatcaccaccatcatcacggtggagcgagcatgactggaggccaacagatgggcagtctgaccacgaatcctggagtgtcagcctggcaggtgaacacagcgtacactgctggccaactcgtgacctacaatggacgcacctatcgcagtctgcaaccgcacacctcacttgcaggctgggaaccgagcaacgttcctgctctgtggcagctccagagcttaggtggctctggcagtggtgacgacgacgatcgtcatgcagaaggcaccttcaccagcgatgtgtccagctatctggaaggtcaagcagcgaaagagttcattgcgtggttagttcgtggtcgtggctaa

SEQ ID NO: 146 - protein, 120 aa, Synthetic construct
MAKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 147 - protein, 120 aa, Synthetic construct
MRKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 148 - protein, 120 aa, Synthetic construct
MNKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 149 - protein, 120 aa, Synthetic construct
MDKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 150 - protein, 120 aa, Synthetic construct
MCKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 151 - protein, 120 aa, Synthetic construct
MQKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 152 - protein, 120 aa, Synthetic construct
MEKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 153 - protein, 120 aa, Synthetic construct
MGKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 154 - protein, 120 aa, Synthetic construct
MHKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 155 - protein, 120 aa, Synthetic construct
MIKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 156 - protein, 120 aa, Synthetic construct
MLKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 157 - protein, 120 aa, Synthetic construct
MKKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 158 - protein, 120 aa, Synthetic construct
MMKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 159 - protein, 120 aa, Synthetic construct
MFKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 160 - protein, 120 aa, Synthetic construct; identical to SEQ ID NO: 147

SEQ ID NO: 161 - protein, 120 aa, Synthetic construct
MPKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 162 - protein, 120 aa, Synthetic construct
MOKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 163 - protein, 120 aa, Synthetic construct
MSKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 164 - protein, 120 aa, Synthetic construct
MUKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 165 - protein, 120 aa, Synthetic construct
MTKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 166 - protein, 120 aa, Synthetic construct
MWKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 167 - protein, 120 aa, Synthetic construct
MYKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 168 - protein, 120 aa, Synthetic construct
MVKYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 169 - protein, 120 aa, Synthetic construct
MANYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 170 - protein, 120 aa, Synthetic construct
MRNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 171 - protein, 120 aa, Synthetic construct
MNNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 172 - protein, 120 aa, Synthetic construct
MDNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 173 - protein, 120 aa, Synthetic construct
MCNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 174 - protein, 120 aa, Synthetic construct
MQNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 175 - protein, 120 aa, Synthetic construct
MENYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 176 - protein, 120 aa, Synthetic construct
MGNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 177 - protein, 120 aa, Synthetic construct
MHNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 178 - protein, 120 aa, Synthetic construct
MINYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 179 - protein, 120 aa, Synthetic construct
MLNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 180 - protein, 120 aa, Synthetic construct
MKNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 181 - protein, 120 aa, Synthetic construct
MMNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 182 - protein, 120 aa, Synthetic construct
MFNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 183 - protein, 120 aa, Synthetic construct; identical to SEQ ID NO: 170

SEQ ID NO: 184 - protein, 120 aa, Synthetic construct
MPNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 185 - protein, 120 aa, Synthetic construct
MONYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 186 - protein, 120 aa, Synthetic construct
MSNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 187 - protein, 120 aa, Synthetic construct
MUNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 188 - protein, 120 aa, Synthetic construct
MTNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 189 - protein, 120 aa, Synthetic construct
MWNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 190 - protein, 120 aa, Synthetic construct
MYNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 191 - protein, 120 aa, Synthetic construct
MVNYHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 192 - protein, 121 aa, Synthetic construct
MAKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 193 - protein, 121 aa, Synthetic construct
MRKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 194 - protein, 121 aa, Synthetic construct
MNKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 195 - protein, 121 aa, Synthetic construct
MDKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 196 - protein, 121 aa, Synthetic construct
MCKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 197 - protein, 121 aa, Synthetic construct
MQKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 198 - protein, 121 aa, Synthetic construct
MEKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 199 - protein, 121 aa, Synthetic construct
MGKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 200 - protein, 121 aa, Synthetic construct
MHKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 201 - protein, 121 aa, Synthetic construct
MIKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 202 - protein, 121 aa, Synthetic construct
MLKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 203 - protein, 121 aa, Synthetic construct
MKKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 204 - protein, 121 aa, Synthetic construct
MMKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 205 - protein, 121 aa, Synthetic construct
MFKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 206 - protein, 121 aa, Synthetic construct; identical to SEQ ID NO: 193

SEQ ID NO: 207 - protein, 121 aa, Synthetic construct
MPKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 208 - protein, 121 aa, Synthetic construct
MOKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 209 - protein, 121 aa, Synthetic construct
MSKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 210 - protein, 121 aa, Synthetic construct
MUKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 211 - protein, 121 aa, Synthetic construct
MTKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 212 - protein, 121 aa, Synthetic construct
MWKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 213 - protein, 121 aa, Synthetic construct
MYKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 214 - protein, 121 aa, Synthetic construct
MVKYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 215 - protein, 121 aa, Synthetic construct
MANYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 216 - protein, 121 aa, Synthetic construct
MRNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 217 - protein, 121 aa, Synthetic construct
MNNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 218 - protein, 121 aa, synthetic construct
MDNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 219 - protein, 121 aa, Synthetic construct
MCNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 220 - protein, 121 aa, Synthetic construct
MQNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 221 - protein, 121 aa, Synthetic construct
MENYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 222 - protein, 121 aa, Synthetic construct
MGNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 223 - protein, 121 aa, Synthetic construct
MHNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 224 - protein, 121 aa, Synthetic construct
MINYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 225 - protein, 121 aa, Synthetic construct
MLNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 226 - protein, 121 aa, Synthetic construct
MKNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 227 - protein, 121 aa, Synthetic construct
MMNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 228 - protein, 121 aa, Synthetic construct
MFNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 229 - protein, 121 aa, Synthetic construct; identical to SEQ ID NO: 216

SEQ ID NO: 230 - protein, 121 aa, Synthetic construct
MPNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 231 - protein, 121 aa, Synthetic construct
MONYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 232 - protein, 121 aa, Synthetic construct
MSNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 233 - protein, 121 aa, Synthetic construct
MUNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 234 - protein, 121 aa, Synthetic construct
MTNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 235 - protein, 121 aa, Synthetic construct
MWNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 236 - protein, 121 aa, Synthetic construct
MYNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 237 - protein, 121 aa, Synthetic construct
MVNYHHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 238 - protein, 121 aa, Synthetic construct
MAKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 239 - protein, 121 aa, Synthetic construct
MRKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 240 - protein, 121 aa, Synthetic construct
MNKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 241 - protein, 121 aa, synthetic construct
MDKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 242 - protein, 121 aa, Synthetic construct
MCKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 243 - protein, 121 aa, Synthetic construct
MQKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 244 - protein, 121 aa, Synthetic construct
MEKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 245 - protein, 121 aa, Synthetic construct
MGKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 246 - protein, 121 aa, Synthetic construct
MHKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 247 - protein, 121 aa, Synthetic construct
MIKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 248 - protein, 121 aa, Synthetic construct
MLKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 249 - protein, 121 aa, Synthetic construct
MKKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 250 - protein, 121 aa, Synthetic construct
MMKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 251 - protein, 121 aa, Synthetic construct
MFKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 252 - protein, 121 aa, Synthetic construct; identical to SEQ ID NO: 239

SEQ ID NO: 253 - protein, 121 aa, Synthetic construct
MPKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 254 - protein, 121 aa, Synthetic construct
MOKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 255 - protein, 121 aa, Synthetic construct
MSKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 256 - protein, 121 aa, Synthetic construct
MUKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 257 - protein, 121 aa, Synthetic construct
MTKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 258 - protein, 121 aa, Synthetic construct
MWKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 259 - protein, 121 aa, Synthetic construct
MYKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 260 - protein, 121 aa, Synthetic construct
MVKYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 261 - protein, 121 aa, Synthetic construct
MANYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 262 - protein, 121 aa, Synthetic construct
MRNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 263 - protein, 121 aa, Synthetic construct
MNNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 264 - protein, 121 aa, Synthetic construct
MDNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 265 - protein, 121 aa, Synthetic construct
MCNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 266 - protein, 121 aa, Synthetic construct
MQNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 267 - protein, 121 aa, Synthetic construct
MENYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 268 - protein, 121 aa, Synthetic construct
MGNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 269 - protein, 121 aa, Synthetic construct
MHNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 270 - protein, 120 aa, Synthetic construct; identical to SEQ ID NO: 178

SEQ ID NO: 271 - protein, 121 aa, Synthetic construct
MLNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 272 - protein, 121 aa, Synthetic construct
MKNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 273 - protein, 121 aa, Synthetic construct
MMNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 274 - protein, 121 aa, Synthetic construct
MFNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 275 - protein, 121 aa, Synthetic construct; identical to SEQ ID NO: 262

SEQ ID NO: 276 - protein, 121 aa, Synthetic construct
MPNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 277 - protein, 121 aa, Synthetic construct
MONYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 278 - protein, 121 aa, Synthetic construct
MSNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 279 - protein, 121 aa, Synthetic construct
MUNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 280 - protein, 121 aa, Synthetic construct
MTNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 281 - protein, 121 aa, Synthetic construct
MWNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 282 - protein, 121 aa, Synthetic construct
MYNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG

SEQ ID NO: 283 - protein, 121 aa, Synthetic construct
MVNYQHHHHHHGGASMTGGQQMGSLTTNPGVSAWQVNTAYTAGQLVTYNGRTYRSLQPHTSLAGWEPSNVPALWQLQSLGGSGSGDDDDRHAEGTFTSDVSSYLEGQAAKEFIAWLVRGRG